(19) United States
(12) Patent Application Publication
(10) Pub. No.: US 2009/0043140 A1
(43) Pub. Date: Feb. 12, 2009

(54) Narrow cut waxes and method of making

(75) Inventors: Hu Yang, Manvel, TX (US); Eduardo J. Baralt, Kingwood, TX (US); Ruthann Marty Hickox, Spring, TX (US); Steve Hutchison, Spring, TX (US)

Correspondence Address: CHEVRON PHILLIPS CHEMICAL COMPANY LP, LAW DEPARTMENT - IP, P.O BOX 4910, THE WOODLANDS, TX, 77387-4910, US

(73) Assignee: Chevron Phillips Chemical Company LP

(21) Appl. No.: 11/891,194

(22) Filed: Aug. 9, 2007

(51) Int. Cl. C07C 5/03

(52) U.S. Cl. 585/277; 585/802; 585/809; 585/812

(57) ABSTRACT

A method of producing a wax composition having a constrained carbon number distribution and/or a narrow melting range is described. Additionally, hydrocarbon waxes and hydrocarbon wax compositions having a constrained carbon number distribution and/or melting point are described.

### BACKGROUND OF THE INVENTION

Waxes have many useful application(s) which depend upon their melting characteristics. Many wax applications only require that the wax has a melting point and/or a melting point within a broad temperature range. However, some wax applications, such as printing inks and investment casting, among others, require a wax which has a melting point within a specific temperature range and/or has a defined melting range. In extreme cases the wax composition must additionally have a narrow melting range. Within a particular class of wax compounds, the melting range is usually dependent upon the carbon number range of compounds present and/or the number of compounds having different structures (e.g. linear versus branched, among others) present in the wax composition. Consequently, the narrower the carbon number range of the molecules, and/or the fewer the number of different structures present in the wax composition, the narrower the melting range of the wax composition.

Many wax compositions having a defined melting point within a specific temperature range are prepared by physical methods which separate molecules based upon their carbon number and/or structure (e.g. distillation, among others). Typically, the compositions having a defined melting point are produced from other compositions having many different compounds with carbon numbers within close proximity to each other and/or similar structures. However, the physical separation methods place limits upon the narrowness of the carbon number range which can be achieved when separating the desired composition from a composition having a large number of compounds with carbon numbers within close proximity to each other. For example, in distillation, higher distillation temperatures reduce the ability to separate the compounds that differ by only a few carbon numbers. Consequently, the higher the melting point of the components of the wax composition obtained by these physical separation methods, the larger the carbon number range and/or the melting range of the resulting wax composition will be. Even using vacuum distillation techniques, which are often used to reduce the temperature of the distillation, does not help. While vacuum distillation will lower the boiling points of the compounds to be separated, the use of vacuum distillation also decreases the difference between the boiling points of the different compounds making them harder to separate. Consequently, using vacuum distillation techniques achieves very little, if any, improvement in decreasing the carbon number range and/or melting range of the resulting wax composition.

What are needed are methods to produce wax compositions having a narrow carbon number distribution and/or a narrow melting range which do not depend upon the physical separation of wax compounds from compositions having a large number of compounds with carbon numbers within close proximity to each other.

Potentially, one method of producing a wax having a narrow carbon number distribution and/or melting range is to prepare the wax from a lower molecular weight material (which may be easily separated from the product) such that the product has a narrow carbon number range and/or melting range. One potential reaction that could be utilized is olefin metathesis.

Olefin metathesis (also sometimes known as olefin disproportionation) is a reaction whereby one or more olefins are transformed into different olefins. Olefin metathesis usually produces product olefins having a different molecular weight than the feedstock olefins wherein the average molecular weight of the metathesis products is the same as the average molecular weight as the feedstock olefins. For example, in the hypothetical absence of olefin isomerization, the self metathesis of 2-octene produces equal molar amounts of 2-butene and 6-dodecene while the metathesis of equal molar amounts of 1-hexene and 3-heptene will produce equal molar amounts of ethylene, 1-butene, 1-pentene, 3-hexene, 3-octene, 4-octene, 4-nonene, and 5-decene.

However, a problem with olefin metathesis is that the metathesis catalyst isomerizes the position of the olefin double bond of the olefin feedstock and the metathesis product. These isomerized olefins (both isomerized olefin feedstock and isomerized metathesis product) can further participate in the metathesis reactions. Consequently, olefin metathesis of mixtures of olefins having different numbers of carbon atoms, and even an olefin feedstock having a single long-chained olefin (e.g. 1-hexecene or 1-octadecene, among others), will produce a complex mixture of olefin products having a broad distribution of carbon numbers.

What are needed are methods to utilize olefin metathesis to produce wax metathesis products having a narrow carbon number distribution and/or narrow melting range.

### SUMMARY OF THE INVENTION

In an aspect, the present invention is directed to a method comprising: 1) contacting a feedstock composition comprising an olefin feedstock comprising, or consisting essentially of, olefins having at least 16 carbon atoms and a catalyst composition comprising a metathesis catalyst, 2) reacting the olefin feedstock at metathesis reaction conditions, and 3) producing a wax metathesis product having a constrained carbon number distribution and/or narrow melting range. In another aspect, the present invention is directed to a method comprising: 1) contacting a feedstock composition comprising an olefin feedstock comprising, or consisting essentially of, olefins having at least 16 carbon atoms and a catalyst composition comprising a metathesis catalyst, and 2) reacting the olefin feedstock at metathesis reaction conditions selected to produce a wax metathesis product having a constrained carbon number distribution and/or narrow melting range. In an embodiment, the method(s) can be a method of controlling the carbon number distribution of a wax metathesis product such that the wax metathesis product has a constrained carbon number distribution and/or a narrow melting range is produced. In another aspect, the present invention is also directed to a method comprising: 1) controlling a carbon number distribution of a wax metathesis product by selecting metathesis reaction parameters including; a) an olefin feedstock comprising, or alternatively, consisting essentially of, olefins having at least 16 carbon atoms, b) a metathesis catalyst, and c) metathesis reaction conditions; 2) contacting a feedstock composition comprising the olefin feedstock and catalyst composition comprising the metathesis catalyst; and 3) reacting the olefin feedstock at the metathesis reaction conditions to produce a wax metathesis product having a constrained carbon number distribution and/or narrow melting range. In an embodiment, the constrained carbon number distribution may be described as a carbon number span. In some embodiments, the carbon number span of the wax metathesis product is greater than 1 and less than 15. In some embodiments, the melting range of the wax metathesis product is less than 15° C. In some embodiments, the olefin feedstock used in the method of the present invention comprises, or consists essentially of, normal alpha olefins. In some embodiments, the metathesis catalyst used for the method of the present invention is a ruthenium carbene metathesis catalyst or a molybdenum carbene metathesis catalyst. In some embodiments, the olefin feedstock used in the method of the present invention has a carbon number span greater than 1 and less than 8 carbon atoms. In some embodiments, the metathesis reaction conditions include a metathesis reaction temperature ranging from the melting point of the olefin feedstock to 120° C. In some embodiments, the olefin feedstock has a carbon number span of 3, 4, or 5, the metathesis catalyst is a metal carbene metathesis catalyst, the metathesis reaction conditions comprise a metathesis reaction temperature ranging from the melting point of the olefin feedstock to 120° C., and the wax metathesis product has a carbon number span ranging from 5 to 14. Other embodiments of the method are provided in the present disclosure.

In an aspect, the present invention is also directed to a wax composition comprising, or consisting essentially of, wax molecules having a constrained carbon number distribution and/or a narrow melting range. In some embodiments, the wax molecules have a) a carbon number span greater than 1 and less than 15, and b) a distribution of wax molecules wherein 85 weight % of the wax molecules have a molecular weight within 49 grams/mole of the average molecular weight of the wax molecules. In other embodiments, the wax molecules have a) greater than 30 carbon atoms, b) a carbon number span greater than 1 and less than 15, and c) a distribution of wax molecules wherein 85 weight % of the wax molecules have a molecular weight within 49 grams/mole of the average molecular weight of the wax molecules. In some embodiments, the melting range of the wax metathesis product is less than 15° C. Other embodiments of the wax composition(s) and/or wax molecule(s) are provided in the present disclosure.

In an embodiment, the wax molecules can be hydrocarbon wax molecules and the wax composition can be a hydrocarbon wax composition comprising, or consisting essentially of, hydrocarbon wax molecules. In an embodiment, the hydrocarbon wax molecules have a constrained carbon number distribution and/or a narrow melting range. In an embodiment the hydrocarbon wax molecules have a) greater than 30 carbon atoms, b) a carbon number span greater than 1 and less than 15, and c) a distribution of hydrocarbon wax molecules wherein 85 weight % of the hydrocarbon wax molecules have a molecular weight within 49 grams/mole of the average molecular weight of the hydrocarbon wax molecules. In some embodiments, the melting range of the hydrocarbon wax molecules is less than 15° C. In some embodiments, the hydrocarbon wax molecules have a DSC melting point greater than 50° C. and a DSC melting range of less than 15° C. In some embodiments, the hydrocarbon wax molecules comprise, or consist essentially of, olefinic hydrocarbon molecules; or alternatively, saturated hydrocarbon wax molecules. In some embodiments, the hydrocarbon wax molecules comprise, or consist essentially of, linear wax molecules. In some embodiments, the hydrocarbon wax molecules comprise, or consist essentially of, olefinic hydrocarbon wax molecules having a DSC melting point ranging from 65° C. to 85° C. and a DSC melting range of less than 15° C.; alternatively, saturated hydrocarbon wax molecules having a DSC melting point ranging from 75 to 95° C. and a DSC melting range of less than 15° C.; alternatively, olefinic hydrocarbon wax molecules having a DSC melting point ranging from 70 to 100° C. and DSC melting range of less than 15° C.; or alternatively, saturated hydrocarbon wax molecules having a DSC melting point ranging from 85 to 105° C. and a DSC melting range of less than 15° C. Other embodiments of the hydrocarbon wax composition(s) and/or hydrocarbon wax molecule(s) are provided in the present disclosure.

### DEFINITIONS

The term “organic group” and its derivatives (e.g. “organyl” or organylene) whenever used in this specification and claims refers to compounds or groups comprising carbon and hydrogen regardless of functional type. Thus, an “organic group” can contain organic functional groups and/or atoms other than carbon and hydrogen. For example, non-limiting examples of atoms other than carbon and hydrogen include halogens, oxygen, nitrogen, and phosphorus, among others. Non-limiting examples of functional groups include ethers, aldehydes, ketones, aldehydes, esters, sulfides, amines, and phosphines, among others. The term “organic group” also includes heteroatom containing rings, heteroatom containing ring systems, heteroaromatic rings, and heteroaromatic ring systems, among others. Finally, it should be noted that the term “organic group” includes “hydrocarbon group” as a member.

The term “hydrocarbon(s)” and its derivatives (e.g. “hydrocarbyl”) whenever used in this specification and claims refer to compounds or groups comprising only hydrogen and carbon. The term “hydrocarbon” may also be prefaced with other descriptors which further limit the scope of the term. For example “olefinic hydrocarbons” refer to compounds or groups containing only hydrogen and carbon and have at least one olefinic double bond, “aromatic hydrocarbon(s)” refer to compound or groups containing only hydrogen and carbon and having an aromatic ring or ring system (i.e. a benzene ring or naphthalene ring system, among others), and “saturated hydrocarbon(s)” refers to compounds or groups containing only hydrogen and carbon and having no olefinic or aromatic double bonds. The term “hydrocarbon(s)” when prefaced with an atom or functional group descriptor indicates that the compound(s) or group(s) contains only hydrogen, carbon, and the indicated atom or functional group. For example, a “halogenated hydrocarbon(s)” refers to a compound(s) containing hydrogen, carbon, and at least one halogen atom but no other type of heteroatom.

The term “wax” whenever used in this specification and claims refers to an organic material having a melting point greater than 35° C. at 1 atmosphere. The term “wax composition” whenever used in this specification and claims refers to a composition comprising a wax or wax molecules. The term “wax composition” may also refer to composition consisting essentially of, or consisting of, a wax or wax molecules when indicated. Compounds which are not wax molecules (e.g. solvents and additives, among others) may be present in the “wax composition” comprising hydrocarbon wax molecules unless otherwise indicated. The term “hydrocarbon wax composition” whenever used in this specification and claims refers to a composition comprising hydrocarbon wax molecules. The term “hydrocarbon wax composition” may also refer to compositions consisting essentially of, or consisting of, a hydrocarbon wax or hydrocarbon wax molecules when indicated. Compounds which are not a hydrocarbon wax molecules (e.g. solvents and additives, among others) may be present in the “hydrocarbon wax composition” comprising hydrocarbon wax molecules unless otherwise indicated.

The term “melting point” or “DSC melting point” whenever used in this specification and claims refers to the melting point as determined by Differential Scanning Calorimetry (hereafter may be referred to as DSC). The “DSC melting point” is determined using ASTM D 3418, using two heating scans with an interceding cooling scan. The first heating scan and second heating scans are conducted with a heating rate of 20° C./min from −20° C. to 120° C. The interceding cooling scan is conducted at rate of 20° C./min 120° C. to −20° C. The “DSC melting point” is taken to be the temperature within the DSC melting transition of the second heating scan having the greatest heat flow.

The term “melting range” or “DSC melting range” whenever used in this specification and claims refers to the difference between the temperature at which the material (e.g. wax metathesis product or wax molecules, among others) starts to melt and the temperature at which melting is complete as measured by Differential Scanning Calorimetry. The “DSC melting range” is determined using ASTM D 3418 using two heating scans with an interceding cooling scan. The first heating scan and second heating scans are conducted with a heating rate of 20° C./min from −20° C. to 120° C. The interceding cooling scan is conducted at rate of 20° C./min 120° C. to −20° C. The “DSC melting range” is taken to be the temperature difference between the onset and end point of the DSC melting transition of the second heating scan as determined by the crossing point between the baseline and normal slope of the DSC melting transition peak.

The term “feedstock olefin(s)” or “olefin feedstock” whenever used in this specification and claims refer to the olefinic compounds which are originally present in the feedstock composition or the olefin feedstock composition before being contacted with the metathesis catalyst. The term “feedstock olefin(s)” or “olefin feedstock” do not include any new olefinic compounds produced during the metathesis reaction (via metathesis and/or isomerization reactions) which were not originally present in the “olefin feedstock.”

The term “metathesis product(s)” whenever used in this specification and claims refers to organic material(s) produced by a metathesis reaction. The term “metathesis product(s)” refers to material(s) produced by a metathesis reaction having a carbon number at least 3 greater than the carbon number of the original feedstock olefin(s) having the greatest number of carbon atoms present in a quantity of at least 3 weight percent, or having a carbon number at least 3 less than the carbon number of the original feedstock olefin(s) having the fewest carbon atoms present in a quantity of at least 3 weight percent. Consequently, the term “metathesis product(s)” excludes compounds which are recreations of the feedstock olefin, have a carbon number the same as a feedstock olefin, or have a carbon number close to that of the feedstock olefin(s) as defined herein.

The term “wax metathesis product(s)” whenever used in this specification and claims refers to the organic material(s) of the metathesis product having a melting point greater than 35° C. at 1 atmosphere. As the term “wax metathesis product(s)” is a subset of the term “metathesis product(s),” the term “wax metathesis product(s)” is subject to the limitations in regards to the materials/compounds considered to constitute a “metathesis product.” Consequently, the term “wax metathesis product” does not include any wax compounds produced (via isomerization and metathesis reactions) which recreate the feedstock olefin(s), have a carbon number the same as a feedstock olefin, or have a carbon number close to that of a feedstock olefin as defined by the term “metathesis product” In addition to the stipulation the material(s) must have a melting point greater than 35° C. at 1 atmosphere.

The term “non-wax metathesis product olefin(s)” whenever used in the specification and claims refers to the olefin(s) which are not wax metathesis products. The term “non-wax metathesis product olefin(s)” encompasses olefins produced during the metathesis (via isomerization and metathesis reactions) which are not “wax metathesis products.” The term “non-wax metathesis product olefin(s) also includes metathesis product olefins which are not a wax. Additionally, the term “non-wax metathesis product olefin(s)” includes feedstock olefin(s).

The term “alpha olefin” whenever used in this specification and claims refers to an olefin that has a double bond between the first and second carbon atom. The term “alpha olefin” includes linear and branched alpha olefins unless expressly stated otherwise. In the case of branched alpha olefins, a branch may be at the 2-position (a vinylidene) and/or the 3-position or higher with respect to the olefin double bond. The term “vinylidene” whenever used in this specification and claims refers to an alpha olefin having a branch at the 2-position with respect to the olefin double bond. The term “alpha olefin,” by itself, does not indicate the presence or absence of heteroatoms and/or the presence or absence of other carbon-carbon double bonds unless explicitly indicated. The term “hydrocarbon alpha olefin” or “alpha olefin hydrocarbon” refers to alpha olefin compounds containing only hydrogen and carbon.

The term “normal alpha olefin” whenever used in this specification and claims refers to a linear hydrocarbon mono-olefin having a double bond between the first and second carbon atom. It should be noted that “normal alpha olefin” is not synonymous with “linear alpha olefin” as the term “linear alpha olefin” can include linear olefinic compounds having a double bond between the first and second carbon atoms and having heteroatoms and/or additional double bonds.

The term “consists essentially of normal alpha olefin(s),” or variations thereof, whenever used in this specification and claims refers to commercially available normal alpha olefin product(s). The commercially available normal alpha olefin product can contain non-normal alpha olefin impurities such as vinylidenes, internal olefins, branched alpha olefins, paraffins, and diolefins, among other impurities, which are not removed during the normal alpha olefin production process. One of ordinary skill in the art will recognize that the identity and quantity of the specific impurities present in the commercial normal alpha olefin product will depend upon the source of commercial normal alpha olefin product. Consequently, the term “consists essentially of normal alpha olefins” and its variants is not intended to limit the amount/quantity of the non-linear alpha olefin components any more stringently than the amounts/quantities present in a particular commercial normal alpha olefin product. One source of commercially available alpha olefins products are those produced by the oligomerization of ethylene. A second source of commercially available alpha olefin products are those which are produced, and optionally isolated from, Fischer-Tropsch synthesis streams One source of commercially available normal alpha olefin products produced by ethylene oligomerization which may be utilized as an olefin feedstock is Chevron Phillips Chemical Company LP, The Woodlands, Tex. Other sources of commercially available normal alpha olefin products produced by ethylene oligomerization which may be utilized as an olefin feedstock include Ineos Oligomers (Feluy, Belgium), Shell Chemicals Corporation (Houston, Tex. or London, United Kingdom), Idemitsu Kosan (Tokyo, Japan), and Mitsubishi Chemical Corporation (Tokyo, Japan) among others. One source of commercially available normal alpha olefin products produced, and optionally isolated from Fisher-Tropsch synthesis streams includes Sasol (Johannesburg, South Africa) among others.

The term “internal olefin(s)” whenever used in this specification and claims refers to an olefin which has a double bond at any position other than between the first and second carbon atom. An “internal olefin(s)” can be linear or branched. A “branched internal olefin” may have a branch attached to one of the carbon atoms of the internal double bond and/or may have a branch at any carbon atom other than those participating in the internal olefin double bond. The term “internal olefin(s)” does not indicate the presence or absence of other groups, branches, heteroatoms, or double bonds within the “internal olefin(s)” unless explicitly indicated. A “disubstituted olefin(s)” whenever used in this specification and claims refers to an olefin(s) having only two organyl groups attached to the carbon atoms of the carbon-carbon double bond. The two organyl groups attached to the carbon atoms of the carbon-carbon double bond of the “disubstituted olefin(s)” may be attached to the same carbon atom (e.g. a vinylidene) or may be attached to different carbon atoms of the carbon-carbon double bond. The term “disubstituted olefin(s)” does not indicate the presence or absence of other groups, heteroatoms, branches, or double bonds within the “disubstituted olefin(s)” unless explicitly indicated. The term “internal disubstituted olefin(s)” whenever used in this specification and claims refers to an olefin(s) having wherein the double bond is not a terminal double bond (e.g. one and only one organyl group attached to each carbon atom of the carbon-carbon double bond). The term “internal disubstituted olefin(s)” does not indicate the presence or absence of other groups, heteroatoms, branches, or double bonds within the “internal disubstituted olefin(s)” unless explicitly indicated. A “linear internal olefin(s)” whenever used in this specification and claims refers to an linear olefin(s) wherein the double bond is not a terminal double bond (e.g. one and only one organyl group attached to each carbon atom of the carbon-carbon double bond). The term “linear internal olefin(s)” does not indicate the presence or absence of other groups, heteroatoms, or double bonds unless explicitly indicated (e.g. mono-olefinic linear internal olefin). A “trisubstituted olefin(s)” whenever used in this specification and claims refers to an internal olefin(s) having three organyl groups attached to the carbon atoms participating in the olefin internal double bond. The term “trisubstituted olefin(s)” does not indicate the presence or absence of other groups, branches, or double bonds within the “trisubstituted olefin(s)” unless explicitly indicated. “Tetrasubstituted olefin(s)” whenever used in this specification and claims refers to an internal olefin(s) having two organyl groups attached to each carbon atom of the olefin double bond. The term “tetrasubstituted olefin(s)” does not indicate the presence or absence of other groups, branches, or double bonds within the “tetrasubstituted olefin(s)” unless explicitly indicated.

The terms “carbon number span,” “span of carbon numbers,” their alternatives, or derivatives whenever used in this specification and claims refer to a contiguous spread of carbon numbers which represents a subset of the compounds having a particular characteristic(s) (e.g. olefin feedstock, wax metathesis product, or hydrocarbon molecules, among others) in a composition. Specifically, the “carbon number span,” or its alternatives and derivatives, is a contiguous spread of carbon numbers wide enough to include all the carbon numbers of the compound(s) having the particular characteristic(s) present in at least 3 weight percent and together the compound(s) represented by the included carbon numbers comprise greater than 95 weight percent of the compound(s) having the particular characteristic(s).

For the purpose of determining the “carbon number span,” when more the one compound having the particular characteristic and having the same carbon number is present, it is the total weight percentage of all the compounds having the particular characteristic and having the same carbon number which is used to determine which carbon numbers are included in the “carbon number span.” For example, if an olefin feedstock composition comprising olefins has 1.4 weight percent 1-octadecene and 2.1 weight percent 2-ethyl-1-hexadecene, the carbon number span must be wide enough to include the carbon number 18 because the carbon number 18 represents 3.5 weight percent of the olefin feedstock.

The term “carbon number span,” or its alternatives, can refer to a particular class of molecules and/or compounds within a composition or mixture as identified by the particular descriptive characteristic(s). In this case, the “carbon number span” is not affected by the carbon number, and respective weight percents, of any compound(s) which does not have the particular descriptive characteristic(s). For example, the “carbon number span of the olefin feedstock” is determined by the carbon number(s), and the respective weight percents, of the olefin feedstock and is not affected the carbon number, and respective weight percent, of any compound(s) (e.g. solvent of diluent) which is not an “olefin feedstock” regardless of whether these other materials are present in a composition comprising olefin feedstock or have a carbon number within or outside the carbon number span of the olefin feedstock. For example, an olefin feedstock having a carbon number span of 5 can refer to a composition consisting essentially of a olefin feedstock consisting of olefins having 20, 22, and 24 carbon atoms or a composition comprising an olefin feedstock consisting of olefins having 4, 5, 6, 7, and 8 carbon atoms regardless of the presence and/or quantity of a saturated C₆ solvent in the composition comprising the olefin feedstock

Numerically, the carbon number span is represented by the equation MC−FC+1 wherein MC is the carbon number of the compound(s) having the greatest number of carbon atoms and FC is the carbon number of the compound(s) having the fewest number of carbon atoms within the contiguous spread of carbon numbers defined above. FC and MC are determined by applying the criteria noted above to the compounds having the particular descriptive characteristic(s). The “carbon number span,” and consequently MC and FC, are not affected by the absence of compounds with a particular carbon number that falls within the contiguous spread of carbon numbers. Furthermore, the “carbon number span,” and consequently MC and FC, are not affected by the presence and/or quantity of compound(s) which do not have the particular descriptive characteristic(s).

As a starting point, MC and FC represents the largest and smallest carbon numbers, respectively, of the compounds having a particular descriptive characteristic(s) present in at least 3 weight percent. However, depending upon the particular composition, it is possible that the carbon number span represented by the largest and smallest carbon numbers of the compounds having a particular descriptive characteristic(s) present in at least 3 weight percent does not include greater than 95 weight percent of the compounds having the particular descriptive characteristic(s). In this latter case, MC and/or FC would be determined by then including the carbon number, and any intervening carbon numbers, of the compounds having the particular descriptive characteristic(s) in order of decreasing weight percent presence until greater than 95 weight percent of the compound(s) having the particular descriptive characteristic(s) are encompassed by the contiguous spread of carbon numbers.

For example, given the olefin feedstock carbon number distribution provided below, the “carbon number span” of the olefin feedstock would be 8 (MC=21, FC=14, MC−FC+1=8). The “carbon number span” for the olefin feedstock is not 6 because only

| | C₁₃ | C₁₄ | C₁₅ | C₁₆ | C₁₇ | C₁₈ | C₁₉ | C₂₀ | C₂₁ | C₂₂ |
|---|---|---|---|---|---|---|---|---|---|---|
| Weight Percent | 1.2 | 4.5 | 11.6 | 24.7 | 27.4 | 19.4 | 5.7 | 1.9 | 2.8 | 0.8 |

only 93.3 weight percent olefin feedstock is encompassed by the contiguous spread of carbon numbers including all the compounds present in at least three weight percent (C₁₄-C₁₉). The “carbon number span” is not 7 even though the contiguous spread of carbon number from C₁₄ to C₂₀ would include 95.2 weight percent of the olefin feedstock because the next olefin feedstock carbon number present in the largest amount is C₂₁, and not C₂₀. Since the carbon number of the olefin feedstock present in the next highest amount is C₂₁, and the contiguous spread of carbon numbers from C₁₄ to C₂₁ encompasses 98.0 weight of the olefin feedstock, the carbon number span of this exemplary composition is 8.

It should be noted that the carbon number span can be 1. For example, if greater than 95 weight percent of compounds having the particular descriptive characteristic(s) have the same carbon number and no other carbon number of compounds having the particular descriptive characteristic(s) is present in at least 3 weight percent, MC and FC are the same and the carbon number span is 1 (i.e., MC−FC+1=1).

The term “having a molecular weight within X grams/mole of the average molecular weight,” or its derivatives, refers to a material(s) meeting the descriptive characteristics of the composition and having a molecular weight ranging from the average molecular weight−X grams/mole to the average molecular weight+X grams/mole. For example, a wax metathesis product defined as having 70 weight percent of the product within 50 grams/mole of a wax metathesis product having an average molecular weight of 500 grams/mole would describe a wax metathesis product wherein 70 weight percent of the wax metathesis product has a molecular weight ranging from 450 to 550 grams/mole. For the purpose of this application average molecular weight and molecular weight may be abbreviated to AMW and MW, respectively.

### DETAILED DESCRIPTION

In an aspect, the present invention relates to methods for producing a wax metathesis product having a constrained carbon number distribution and/or having a narrow melting range among other properties. In another aspect, the present invention also relates to methods for controlling the carbon number distribution of a wax metathesis product such that a wax metathesis product having a constrained carbon number distribution and/or a narrow melting range is produced. In a further aspect, present invention also relates to a wax composition comprising, or consisting essentially of, wax molecules having a constrained carbon number distribution and/or a narrow melting range (among other properties). In yet another aspect, the present invention also relates to a hydrocarbon wax composition comprising, or consisting essentially of, a hydrocarbon wax molecules having a constrained carbon number distribution and/or narrow melting range.

In one aspect, the method comprises: 1) contacting a feedstock composition comprising an olefin feedstock and a catalyst composition comprising a metathesis catalyst, 2) reacting the olefin feedstock at metathesis reaction conditions, and 3) producing a wax metathesis product having a constrained carbon number distribution and/or narrow melting range. In a second aspect, the method comprises: 1) contacting a feedstock composition comprising an olefin feedstock and a catalyst composition comprising a metathesis catalyst, and 2) reacting the olefin feedstock at metathesis reaction conditions selected to produce a wax metathesis product having a constrained carbon number distribution and/or having a narrow melting range. In an embodiment, the method is utilized to control a carbon number distribution and/or melting range of a wax metathesis product such that a wax metathesis product having a constrained carbon number distribution and/or narrow melting range is produced. In a third aspect, the method comprises: 1) controlling a carbon number distribution and/or melting range of a wax metathesis product by selecting metathesis reaction parameters including: a) an olefin feedstock, b) a metathesis catalyst, and c) metathesis reaction conditions; 2) contacting a feedstock composition comprising the olefin feedstock and a catalyst composition comprising the metathesis catalyst; and 3) reacting the olefin feedstock at the metathesis reaction conditions to produce a wax metathesis product having a constrained carbon number distribution and/or having a narrow melting range.

Features of the process(es)/methods such as the olefin feedstock, feedstock composition comprising the olefin feedstock, constraints on the olefins of the olefin feedstock (if any), metathesis catalyst, catalyst composition comprising the metathesis catalyst, constraints of the metathesis catalyst (if any), the wax metathesis product, constraints for the wax metathesis product (e.g. carbon number distribution and/or melting parameters, among others), metathesis reaction conditions, constraints on the metathesis reaction conditions, and other method features and/or steps are independently described herein. These features can be utilized in any combination necessary to describe the process(es)/method(s) to produce the wax metathesis product having the desired constrained carbon number distribution and/or narrow melting range.

Generally, the olefin feedstock can comprise, or consist essentially of, any olefinic compound. Further features that can be utilized to describe the olefins may include the type of olefins present, the carbon number of the olefins present, the carbon number distribution of the olefins, the average molecular weight of the olefin(s), the carbon number span of the olefins, and/or the content of a particular type(s) of olefins present (i.e. weight percent or mole percent), among other olefin feedstock features described herein. These features of the olefin feedstock are independently described herein and may be utilized in any combination to describe the olefins of the olefin feedstock.

In an embodiment, the olefins of the olefin feedstock can comprise, or alternatively consist of aliphatic olefins, aromatic olefins, or combinations thereof; alternatively, aliphatic olefins; or alternatively aromatic olefins. In some embodiments, the olefins of the olefin feedstock (whether aliphatic or aromatic) can comprise, or consist essentially of, linear olefins, branched olefins, or combinations thereof, alternatively, linear olefins; or alternatively, branched olefins. In other embodiments, the olefins of the olefin feedstock (whether aliphatic or aromatic, linear or branched, or combinations thereof) can comprise, or consist essentially of acyclic olefins, cyclic olefins, or combinations thereof; alternatively, acyclic olefins, alternatively, cyclic olefins. In some embodiments, the olefins of the olefin feedstock (whether aliphatic or aromatic, linear or branched, cyclic or acyclic, or combinations thereof) may comprise, or consist essentially of, hydrocarbon olefins. In an embodiment, the olefins of the olefin feedstock (whether aliphatic or aromatic, linear or branched, acyclic or cyclic, hydrocarbon, or combinations thereof) may comprise, or consist essentially of, alpha olefins. In some embodiments, the olefins of the olefin feedstock (whether aliphatic or aromatic, acyclic or cyclic, hydrocarbon, or combinations thereof) may comprise, or consists essentially of, linear alpha olefins and branched alpha olefins wherein the branch(s) resides on a carbon atom at the 3-position or higher with respect to the olefin double bond; alternatively, linear alpha olefins; or alternatively, branched alpha olefins having the branch(s) at the 3-position or higher with respect to the olefin double bond. In an embodiment, the olefins of the olefin feedstock (whether aliphatic or aromatic, linear or branched, acyclic or cyclic, hydrocarbon, or combinations thereof) may comprise, or consists essentially of, hydrocarbon alpha olefins. In an embodiment, the olefins of the olefin feedstock may comprise, or consists essentially of, linear hydrocarbon alpha olefins; or alternatively, normal alpha olefins.

In an embodiment, the olefins of the olefin feedstock (whether aliphatic or aromatic, linear or branched, cyclic or acyclic, hydrocarbon, alpha olefin, or any combination thereof) may comprise, or consist essentially of, mono-olefins. In some embodiments, the olefins of the olefin feedstock (whether aliphatic or aromatic, linear or branched, cyclic or acyclic, alpha olefin, or any combination thereof) may comprise, or consist essentially of, hydrocarbon mono-olefins. In some embodiments, the olefins of the olefin feedstock (whether aliphatic or aromatic, linear or branched, cyclic or acyclic, hydrocarbon, or alpha olefin, normal alpha olefin, mono-olefin, or combinations thereof) may comprises, or consists essentially of, olefins having an even number of carbon atoms; alternatively an odd number of carbon atoms.

In an embodiment, the olefin feedstock may comprise, or consist essentially of, olefins having at least 16 carbon atoms; alternatively, at least 18 carbon atoms; or alternatively, at least 20 carbon atoms. In some embodiments, the olefin feedstock may comprise, or consist essentially of, olefins having from 16 to 50 carbon atoms; alternatively, from 16 to 30 carbon atoms; alternatively, from 16 to 20 carbon atoms; alternatively, from 16 to 18 carbon atoms; alternatively, from 20 to 30 carbon atoms; alternatively, from 20 to 24 carbon atoms; alternatively, from 26 to 28 carbon atoms; alternatively, from 26 to 50 carbon atoms; or alternatively, from 18 to 40 carbon atoms.

In an embodiment, the olefin feedstock has a carbon number span less than 10; alternatively, less than 8; alternatively, less than 6; or alternatively, less than 4. In some embodiments, the olefin feedstock has a carbon number span greater than 1; alternatively, greater than 2, or alternatively, greater than 3. In some embodiments, the olefin feedstock has a carbon number span greater than 1 and less than 10; alternatively, greater than 1 and less than 8; alternatively, greater than 1 and less than 6; alternatively, greater than 1 and less than 4; alternatively, greater than 2 and less than 10; alternatively, greater than 2 and less than 8; alternatively, greater than 2 and less than 6; alternatively, greater than 3 and less than 10; alternatively, greater than 3 and less than 8; or alternatively, greater than 3 and less than 6. In some embodiments, the olefin feedstock has a carbon number span of 3, 4, or 5. In other embodiments, the olefin feedstock has a carbon number span of 1; alternatively, 2; alternatively, 3; alternatively, 4; or alternatively, 5.

In an embodiment, the olefin feedstock can comprise, or consist essentially of, any olefin type described herein, can have any carbon number described herein, and/or any carbon number span described herein. In some exemplary non-limiting combinations, the olefin feedstock has a carbon number span less than 8 and comprises, or consists essentially of, olefins having at least 16 carbon atoms; alternatively, has a carbon number span greater than 1 and less than 8 and comprises, or consists essentially of, olefins having at least 16 carbon atoms; alternatively, has a carbon number span less than 8 and comprises, or consists essentially of, olefins having from 16 to 30 carbon atoms; alternatively, has a carbon number span greater than 1 and less than 6 and comprises, or consists essentially of, olefins having from 16 to 30 carbon atoms; alternatively, has a carbon number span greater than 1 and less than 4 and comprises, or consists essentially of, olefins having from 16 to 30 carbon atoms; alternatively, has a carbon number span greater than 2 and less than 6 and comprises, or consists essentially of, olefins having from 16 to 30 carbon atoms; alternatively, has a carbon number span of 3 and comprises, or consists essentially of, olefins having from 16 to 30 carbon atoms; alternatively, has a carbon number span of 4 and comprises, or consists essentially of, olefins having from 16 to 30 carbon atoms; alternatively, has a carbon number span of 5 and comprises, or consists essentially of, olefins having from 16 to 30 carbon atoms; alternatively, has a carbon number span less than 8 and comprises, or consists essentially of, olefins having from 20 to 30 carbon atoms; alternatively, has a carbon number span greater than 1 and less than 8 and comprises, or consists essentially of, olefins having from 20 to 30 carbon atoms; alternatively, has a carbon number span less than 6 and comprises, or consists essentially of, olefins having from 20 to 30 carbon atoms; alternatively, has a carbon number span greater than 1 and less than 6 and comprises, or consists essentially of, olefins having from 20 to 30 carbon atoms; alternatively, has a carbon number span less than 4 and comprises, or consists essentially of, olefins having from 20 to 30 carbon atoms; alternatively, has a carbon number span greater than 1 and less than 4 and comprises, or consists essentially of, olefins having from 20 to 30 carbon atoms; alternatively, has a carbon number span greater than 2 and less than 6 and comprises, or consists essentially of, olefins having from 20 to 30 carbon atoms; alternatively, has a carbon number span of 3 and comprises, or consists essentially of, olefins having from 20 to 30 carbon atoms; alternatively, has a carbon number span of 4 and comprises, or consists essentially of, olefins having from 20 to 30 carbon atoms; or alternatively, has a carbon number span of 5 and comprises, or consists essentially of, olefins having from 20 to 30 carbon atoms.

In an embodiment, the olefin feedstock can comprise olefins, a certain percentage of which have carbon numbers within a certain carbon number range. In some embodiments, the olefin feedstock can comprise olefins wherein greater than 70 weight percent of the olefins have from 20 to 24 carbon atoms; alternatively, wherein greater than 80 weight percent of the olefins have from 20 to 24 carbon atoms; alternatively, wherein greater than 85 weight percent of the olefins have from 20 to 24 carbon atoms; alternatively, wherein greater than 90 weight percent of the olefins have from 20 to 24 carbon atoms; or alternatively, greater than 95 weight percent of the olefins have from 20 to 24 carbon atoms. In other embodiments, the olefin feedstock comprises olefins wherein greater than 70 weight percent of the olefins have from 24 to 28 carbon atoms; alternatively, wherein greater than 80 weight percent of the olefins have from 24 to 28 carbon atoms; or alternatively, wherein greater than 90 weight percent of the olefins have from 24 to 28 carbon atoms. In yet other embodiments, the olefin feedstock comprises olefins wherein greater than 70 weight percent of the olefins have from 26 to 28 carbon atoms; alternatively, wherein greater than 80 weight percent of the olefins have from 26 to 28 carbon atoms; or alternatively, wherein greater than 90 weight percent of the olefins have from 26 to 28 carbon atoms. In further embodiments, the olefin feedstock comprises olefins wherein greater than 70 weight percent of the olefins have from 28 to 56 carbon atoms; alternatively, wherein greater than 80 weight percent of the olefins have from 28 to 56 carbon atoms; or alternatively, wherein greater than 90 weight percent of the olefins have from 28 to 56 carbon atoms. In an embodiment, the olefin feedstock can consists essentially of olefins having from 20 to 30 carbon atoms; alternatively, from 20 to 24 carbon atoms; alternatively, from 24 to 28 carbon atoms; alternatively, from 26 to 28 carbon atoms; alternatively, from 28 to 60 carbon atoms.

In an embodiment, the olefin feedstock can have a particular average molecular weight; or alternatively, have a particular average molecular weight and a particular carbon number span. Applicable carbon number spans for the olefin feedstock are provided herein and can be utilized in combination with any average molecular weight of the olefin feedstock described herein. In an embodiment, the olefin feedstock can have an average olefin molecular weight greater than 210 grams/mole; alternatively, greater than 240 grams/mole; alternatively, greater than 260 grams/mole; alternatively, greater than 330 grams/mole; or alternatively, greater than 450 grams/mole. In some embodiments, the olefin feedstock can have an average olefin molecular weight ranging from 210 grams/mole to 550 grams/mole; alternatively, ranging from 240 grams/mole to 500 grams/mole; or alternatively, ranging from 270 grams/mole to 450 grams/mole. In other embodiments, the olefin feedstock can have an average olefin molecular weight ranging from 210 grams/mole to 390 grams/mole; alternatively, ranging from 260 grams/mole to 340 grams/mole; alternatively, ranging from 280 grams/mole to 320 grams/mole; or alternatively, ranging from 285 grams/mole to 310 grams/mole. In another embodiment, the olefin feedstock can have an average olefin molecular weight ranging from 330 grams/mole to 420 grams/mole; alternatively, ranging from 350 grams/mole to 400 grams/mole; or alternatively, ranging from 360 grams/mole to 390 grams/mole. In yet another embodiment, the olefin feedstock can have an average olefin molecular weight ranging from 440 grams/mole to 550 grams/mole; alternatively, ranging from 460 grams/mole to 530 grams/mole; or alternatively, ranging from 480 grams/mole to 510 grams/mole. In further embodiments, the olefin feedstock can have an average olefin molecular weight ranging from 480 grams/mole to 700 grams/mole; alternatively, ranging from 500 grams/mole to 640 grams/mole; or alternatively, ranging from 500 grams/mole to 580 grams/mole.

In an embodiment, the olefin feedstock can comprise greater than 30 mole percent alpha olefins. In some embodiments, the olefin feedstock can comprise greater than 45 mole percent alpha olefins; alternatively greater than 60 mole percent alpha olefins; alternatively, greater than 75 mole percent alpha olefins; alternatively, greater than 90 mole percent alpha olefins; or alternatively, greater than 95 mole percent alpha olefins. In other embodiments, the olefin feedstock can comprise from 50 to 99 mole percent alpha olefins; alternatively, 55 to 98 mole percent alpha olefins; alternatively, 60 to 97 mole percent alpha olefins; or alternatively, from 65 to 95 mole percent alpha olefins. In some embodiments, the alpha olefins can be linear alpha olefins and branched alpha olefins wherein the branch(s) resides on a carbon atom at the 3 position or higher; alternatively, linear alpha olefins; alternatively, branched alpha olefins wherein the branch(s) resides on a carbon atom at the 3 position or higher; or alternatively, normal olefins.

In an embodiment, the olefin feedstock can comprise linear alpha olefins. In some embodiments, the olefin feedstock can comprise greater than 30 mole percent linear alpha olefins; alternatively, greater than 45 mole percent linear alpha olefins; alternatively, greater than 60 mole percent linear alpha olefins; or alternatively, greater than 75 mole percent linear alpha olefins. In other embodiments, the olefin feedstock can comprise from 30 to 99 mole percent linear alpha olefins; alternatively, from 40 to 95 mole percent linear alpha olefins; or alternatively, from 50 to 90 mole percent linear alpha olefins.

In an embodiment, the olefin feedstock can comprise, or consist essentially of, a normal alpha olefin. Suitable normal alpha olefins include those produced by ethylene oligomerization and/or by cracking heavy waxes (e.g. Fischer-Tropsch waxes). In some embodiments, the olefin feedstock can comprise, or consists essentially of, normal alpha olefin wax. One source of normal alpha olefin waxes is Chevron Phillips Chemical Company LP, The Woodlands, Tex. Potential commercially available normal alpha olefin include, but are not necessarily limited to 1-hexadecene, 1-octadecene, Alpha Olefin C₂₀₋₂₄, Alpha Olefin C₂₆₋₂₈, and/or Alpha Olefin C_{30+HA}. The normal alpha olefin may also be a Fischer-Tropsch product comprising a mixture of paraffin(s) and olefin(s) wherein the olefins meet the olefin feedstock parameters described herein. One source of Fischer-Tropsch waxes is Sasol, Johannesburg, South Africa. In an embodiment, the olefin feedstock can comprise, or consist essentially of, a mixture of commercially available normal alpha olefins.

Minimally, the feedstock composition comprising the olefin feedstock can comprise any desired olefin feedstock described herein. In an embodiment, the feedstock composition comprising the olefin feedstock can further comprise a solvent or diluent. In some embodiments, the feedstock composition comprising the olefin feedstock can consist essentially of any olefin feedstock described herein; alternatively consists essentially of any olefin feedstock described herein and any solvent or diluent described herein. Solvents or diluents which may be utilized in the feedstock composition comprising the olefinic feedstock are described herein. In other embodiments, the feedstock composition comprising the olefin feedstock can be substantially devoid of solvent or diluent.

Generally one can use any metathesis catalyst that is capable of producing a wax metathesis product having the desired constrained carbon number distribution and/or narrow melting range. However, depending upon other metathesis reaction parameters (e.g. olefin feedstock characteristics and/or the metathesis reaction conditions), particular class(es) of metathesis catalyst(s) may be favored in particular instances.

Metathesis catalysts and/or metathesis catalyst systems can be classified by the compound(s) of the metathesis catalyst contacted with the olefin feedstock or the recognized initial metathesis catalyst species. Often the true catalytic species may be difficult to determine because the initial catalytic specie is transformed to other species once the metathesis reaction is initiated. One of ordinary skill in the art will readily recognize the type of metathesis catalyst or metathesis catalyst system based upon the compound(s) contacted with olefin feedstock. In some instances, one may initially contact a precursor of the metathesis catalyst or metathesis catalyst system to the olefin feedstock and create the metathesis catalyst or metathesis catalyst system in situ. One of skill in the art will readily recognize the particular type of metathesis catalyst and/or metathesis catalyst system from the materials added/contacted with the olefin feedstock.

In an embodiment, the metathesis catalyst can be a metal oxide based metathesis catalyst system, a metal halide based metathesis catalyst system, or a metal carbene based metathesis catalyst system. In some embodiments, the metathesis catalyst can be a metal oxide based metathesis catalyst system or a metal halide based metathesis catalyst system. In other embodiments, the metathesis catalyst may be a metal oxide based metathesis catalyst system; alternatively, a metal halide based metathesis catalyst system; or alternatively, a metal carbene based metathesis catalyst system.

Examples of suitable metal oxide based metathesis catalysts or catalyst systems (hereafter referred to as “metal oxide metathesis catalyst(s)”) can comprise cobalt oxide, molybdenum oxide, tungsten oxide, rhenium oxide, or combinations thereof. In some embodiments, the metal oxide metathesis catalyst further comprises a support; alternatively, the metal oxide metathesis catalyst may be unsupported. Suitable metal oxide metathesis catalyst supports include alumina, silica, silica-alumina, and aluminum-phosphate. In further embodiments, the metal oxide metathesis catalyst further comprises a metal alkyl activator. Suitable metal alkyl activators for the metal oxide metathesis catalyst are described herein. Non-limiting examples of suitable metal oxide metathesis catalysts include molybdenum oxide on alumina (MoO₃/Al₂O₃), tungsten oxide on silica (WO₃/SiO₂), rhenium oxide on alumina (Re₂O₇/Al₂O₃), cobalt oxide and molybdenum oxide on alumina (CoO/MoO₃/Al₂O₃), and rhenium oxide on alumina activated with tetramethyl tin (Re₂O₇/Al₂O₃/SnMe₄). Other suitable metal oxide metathesis catalysts are known to those skilled in the art.

Suitable metal halide based metathesis catalysts and/or catalyst systems (hereafter referred to as “metal halide metathesis catalyst(s)”) can comprise a halide of tungsten, molybdenum, or a mixture thereof. In an embodiment, the halide of the metal halide metathesis catalyst can be chloride, bromide, or iodide; alternatively, chloride; alternatively, bromide; or alternatively, iodide. In some embodiments, the metal halide metathesis catalyst can comprise tungsten chloride, molybdenum chloride, or mixtures thereof. In other embodiments, the metal halide metathesis catalyst comprises tungsten chloride; or alternatively, molybdenum chloride. Typically, the metal halide metathesis catalyst further comprises a metal alkyl activator. Suitable metal alkyl activators for the metal halide metathesis catalyst are described herein. The metal halide metathesis catalyst can further comprise other agents in addition to the metal halide and metal alkyl activator, for example alcohol or oxygen, to provide and/or increase metathesis activity. Non-limiting examples of metal halide metathesis catalysts include tungsten chloride/tetrabutyl tin (WCl₆/SnMe₄), tungsten chloride/ethylaluminum dichloride (WCl₆/EtAlCl₂), tungsten chloride/ethyl-aluminum dichloride/ethyl alcohol (WCl₆/EtAlCl₂/EtOH), molybdenum chloride/triethyl aluminum (MoCl₅/AlEt₃), and molybdenum chloride/triethyl aluminum/O₂ (MoCl₅/AlEt₃/O₂). Other suitable metal halide metathesis catalysts are known to those skilled in the art.

Typically, the metal alkyl activator for the metal oxide metathesis catalysts or the metal halide metathesis catalysts can comprise, or consist essentially of, any metal alkyl. Suitable metal alkyl compounds can include alkyl lithium, alkyl magnesium, alkyl aluminum, alkyl tin compounds, and mixtures thereof. In some embodiments, the metal alkyl compound can be an alkyl lithium compound; alternatively an alkyl magnesium compound; alternatively, an alkyl aluminum compound; or alternatively, an alkyl tin compound. Suitable alkyl aluminum compounds can include trialkyl aluminum compounds and/or alkyl aluminum halide compounds. Suitable alkyl groups for the metal alkyl include any C₁ to C₁₀ hydrocarbyl group; or alternatively, C₁ to C₅ hydrocarbyl group. In some embodiments, the alkyl group for the metal alkyl can be a methyl group, ethyl group, n-propyl group, iso-propyl group, n-butyl group, sec-butyl group, or tert-butyl group; alternatively, a methyl group, ethyl group, n-butyl group, sec-butyl group, or tert-butyl group; alternatively, a methyl group; alternatively, an ethyl group; alternatively, an n-butyl group; alternatively, a sec-butyl group; or alternatively, a tert-butyl group. Examples of suitable trialkyl aluminum compound include trimethyl aluminum, triethyl aluminum, and tributyl aluminum. The halide of the alkyl aluminum halide compounds can be can be chloride, bromide, or iodide; alternatively, chloride; alternatively, bromide; or alternatively, iodide. Examples of suitable alkyl aluminum halides include ethyl aluminum dichloride, diethylaluminum chloride, and ethylaluminum sesquichloride. Suitable alkyl tin compounds include tetramethyl tin, tetraethyl tin, and tetrabutyl tin.

Metal carbene metathesis catalysts and/or catalyst systems (hereafter referred to as “metal carbene metathesis catalyst(s)”) are characterized by the presence of a metal-carbon double bond. As opposed to the metal oxide and the metal halide metathesis catalysts, the metal carbene metathesis catalysts are compounds which have a stable metal-carbon double bond or can form a metal-carbon double bond in situ from a metal precursor having a stable metal-carbon single bond.

The metal of suitable metal carbene metathesis catalysts can comprise, or consist essentially of, tungsten, tantalum, osmium, molybdenum, or ruthenium. In an embodiment, the metal carbene metathesis catalyst can be a tungsten carbene metathesis catalyst, a molybdenum carbene metathesis catalyst, or a ruthenium carbene metathesis catalyst; or alternatively, a ruthenium carbene metathesis catalysts or molybdenum carbene metathesis catalyst. In some embodiments, the metal carbene metathesis catalyst can be a tungsten carbene metathesis catalyst; alternatively, an osmium carbene metathesis catalyst; alternatively, a ruthenium carbene metathesis catalyst; or alternatively, a molybdenum carbene metathesis catalyst.

In an embodiment, the ruthenium carbene metathesis catalyst can have the structure L¹L²X₂Ru═CHR wherein L¹ and L² can be an organic ligand, X is a halide, and R represents hydrogen or a hydrocarbyl group. Further embodiments of the groups L¹, L², X, and R are independently described herein. Generally, the ruthenium carbene metathesis catalyst having the structure L¹L²X₂Ru═CHR can be described using any combination of L¹ described herein, L² described herein, X described herein, and R described herein.

In an embodiment, L¹ and L² of the ruthenium carbene metathesis catalyst having the structure L¹L²X₂Ru═CHR can independently be R′₃P, an imidazolinylidene group, or an imidazolidinylidene group. In some embodiments, L¹ and L² are R′₃P; alternatively, L¹ is R′₃P and L² is an imidazolinylidene group, or an imidazolidinylidene group; alternatively, L¹ is R′₃P and L² is an imidazolinylidene group; alternatively, L¹ is R′₃P and L² is an imidazolidinylidene group; alternatively, L¹ and L² are imidazolinylidene groups; or alternatively, L¹ and L² are imidazolidinylidene groups.

In an embodiment, R′ of R′₃P can be a hydrocarbyl group. In some embodiments, each R′ of R′₃P can be the same; alternatively, each R′ of R′₃P can be different; or alternatively, one R′ of R′₃P can be different from the other two R's. In some embodiments, each R′ of R′₃P can be a C₁ to C₁₅ hydrocarbyl group; or alternatively, a C₁ to C₁₀ hydrocarbyl group. In other embodiments, each hydrocarbyl R′ of R′₃P can independently be an alkyl group or an aromatic group; alternatively, an alkyl group; or alternatively, an aromatic group. In an embodiment each alkyl R′ of R′₃P can independently be a methyl group, ethyl group, n-propyl group, isopropyl group, tert-butyl group, neo-pentyl group, cyclopentyl group, or cyclohexyl group. In some embodiments, one or more R's of R′₃P can be a phenyl group; or alternatively a substituted phenyl group. In an embodiment, the substituents of the substituted phenyl group(s) within R′₃P can be a C₁-C₅ organyl group(s); or alternatively, C₁-C₅ hydrocarbyl group(s). In some embodiments, R′₃P can be a trialkyl phosphine or triphenyl phosphine; alternatively, trialkyl phosphine; or alternatively, triphenyl phosphine. In an embodiment, R′₃P can be trimethyl phosphine, triethyl phosphine, triisopropyl phosphine, tri-tert-butyl phosphine, tri-neopentyl phosphine, tricyclopentyl phosphine, tricyclohexyl phosphine, or triphenyl phosphine; alternatively, triisopropyl phosphine, tri-tert-butyl phosphine, tri-neopentyl phosphine, tricyclopentyl phosphine, tricyclohexyl phosphine, or triphenyl phosphine; alternatively, tricyclopentyl phosphine, tricyclohexyl phosphine, or triphenyl phosphine; alternatively, tricyclopentyl phosphine or tricyclohexyl phosphine; alternatively, tricyclopentyl phosphine; alternatively, tricyclohexyl phosphine; or alternatively triphenyl phosphine.

In an embodiment, the imidazolinylidene group or imidazolidinylidene group can be a C₃ to C₈₀ imidazolinylidene group or imidazolidinylidene group; alternatively, a C₃ to C₅₀ imidazolinylidene group or imidazolidinylidene group; alternatively, a C₅ to C₄₀ imidazolinylidene group or imidazolidinylidene group. In some embodiments, the imidazolinylidene group may be a 1,3-disubstituted imidazolinylidene group. In some embodiments, the imidazolidinylidene group may be a 1,3-disubstituted imidazolidinylidene group. In an embodiment, each 1,3-substitutents of the 1,3-disubstituted imidazolinylidene group or 1,3-disubstituted imidazolidinylidene group can be a hydrocarbyl group. In an embodiment, the 1,3-substitutents of the 1,3-disubstituted imidazolinylidene group or 1,3-disubstituted imidazolidinylidene group can be a C₁ to C₃₀ hydrocarbyl group. In some embodiments, each 1,3-substitutent of the 1,3-disubstituted imidazolinylidene group or 1,3-disubstituted imidazolidinylidene group can independently be a C₆ to C₂₀ aromatic group or a C₁ to C₁₀ alkyl groups. In other embodiments, the 1,3-substitutents of the 1,3-disubstituted imidazolinylidene group or 1,3-disubstituted imidazolidinylidene group can be C₆ to C₂₀ aromatic groups; or alternatively, C₁ to C₁₀ alkyl groups. In an embodiment, the aromatic group(s) of the 1,3-disubstituted imidazolinylidene group or 1,3-disubstituted imidazolidinylidene group can be a substituted aromatic group. In some embodiments, the substituted aromatic group of the 1,3-disubstituted imidazolinylidene group or 1,3-disubstituted imidazolidinylidene group can be a 2-disubstituted phenyl group, a 2,6-disubstituted phenyl group, or, a 2,4,6-trisubstituted phenyl group; alternatively, a 2,6-disubstituted phenyl group; or alternatively, a 2,4,6-trisubstituted phenyl group. Suitable substituents for the substituted phenyl group(s) within the 1,3-disubstituted imidazolinylidene group or 1,3-disubstituted imidazolidinylidene group include any C₁ to C₁₀ hydrocarbyl group; or alternatively, any C₁ to C₅ hydrocarbyl group. In some embodiments, each hydrocarbyl group(s) of the substituted phenyl group(s) within the 1,3-disubstituted imidazolinylidene group or 1,3-disubstituted imidazolidinylidene group can independently be a methyl group, ethyl group, n-propyl group, iso-propyl group, n-butyl group, sec-butyl group, or tert-butyl group; altematively, a methyl group, ethyl group, n-butyl group, sec-butyl group, or tert-butyl group; alternatively, a methyl group; alternatively, an ethyl group, alternatively, an isopropyl group; or alternatively, a tert-butyl group. In some embodiments, the 1,3-substitutents of the 1,3-disubstituted imidazolinylidene group or 1,3-disubstituted imidazolidinylidene group can be a 2,6-diisopropylphenyl group or a 2,4,6-trimethylphenyl group; alternatively, a 2,6-diisopropylphenyl group; or alternatively, a 2,4,6-trimethylphenyl group.

In an embodiment, each X of the ruthenium carbene metathesis catalyst having the structure L¹L²X₂Ru═CHR can independently be chloride, bromide, or iodide. In some embodiments, X can be chloride; alternatively, bromide; or alternatively iodide.

In an embodiment, R of the ruthenium carbene metathesis catalyst having the structure L¹L²X₂Ru═CHR can be hydrogen or a C₁ to C₂₀ hydrocarbyl group; or alternatively, a C₁ to C₂₀ hydrocarbyl group. In some embodiments, hydrocarbyl group R can be a methyl group (—CH₃), an ethyl group (—CH₂CH₃), an isopropyl group (—CH(CH₃)₂), a tert-butyl group (—C(CH₃)₃), a phenyl group (—C₆H₅), a 2-methyl-2-propene group (—CH═C(CH₃)₂), or a 2,2-diphenylethene group (—CH═C(C₆H₅)₂). In other embodiments, R can be a tert-butyl group (—C(CH₃)₃, a phenyl group (—C₆H₅), a 2-methyl-2-propene group (—CH═C(CH₃)₂), or a 2,2-diphenylethene group (—CH═C(C₆H₅)₂); alternatively, hydrogen; alternatively, a tert-butyl group (—C(CH₃)₃); alternatively, a phenyl group (—C₆H₅); alternatively, a tert-butyl group (—C(CH₃)₃); alternatively, a phenyl group (—C₆H₅); alternatively, a 2-methyl-2-propene group (—CH═C(CH₃)₂); or alternatively, a 2,2-diphenylethene group (—CH═C(C₆H₅)₂).

In some non-limiting embodiments, the ruthenium carbene metathesis catalyst can be dichloro(phenylmethylene)bis(tricyclohexyl phosphine)ruthenium, dichloro(3-methyl-2-butenylidene) bis(tricyclohexyl phosphine)ruthenium, dichloro(3-methyl-2-butenylidene)bis(tricyclopentyl phosphine) ruthenium, 1,3-bis-(2,4,6-trimethylphenyl)-2-(imidazolidinyl-idene)(phenylmethylene)dichloro(tricyclohexyl phosphine)ruthenium, or 1,3-bis-(2,6-diiso-propylphenyl)-2-(imidazolidinylidene)(phenylmethylene)dichloro(tricyclohexyl phosphine)ruthenium. In some embodiments, the ruthenium metal carbene metathesis catalyst can be di-chloro(phenylmethylene)bis(tricyclohexyl phosphine)ruthenium; alternatively, dichloro(3-methyl-2-butenylidene)bis(tricyclohexyl phosphine)ruthenium; alternatively, 1,3-bis-(2,4,6-trimethylphenyl)-2-(imidazolidinylidene)(phenylmethylene)dichloro(tricyclohexyl phosphine)ruthenium; or alternatively, 1,3-bis-(2,6-diisopropylphenyl)-2-(imidazolidinyl-idene)(phenylmethylene)dichloro(tricyclohexyl phosphine)ruthenium.

In an embodiment, the molybdenum carbene metathesis catalyst can have the structure Mo(═CHR)(NAr)(OR′)₂ wherein R is a hydrogen or hydrocarbyl group, Ar is a substituted aromatic ring, and R′ is a hydrocarbyl group or a halogenated hydrocarbyl group. Further embodiments of the groups R, Ar and R′ are independently described herein. Generally, the molybdenum carbene metathesis catalyst having the structure Mo(═CHR)(NAr)(OR′)₂ can be described using any combination of R described herein, Ar described herein, and R′ described herein.

In some embodiments, R of the molybdenum carbene metathesis catalyst having the structure Mo(═CHR)(NAr)(OR′)₂ can be hydrogen or a C₁ to C₂₀ hydrocarbyl group; or alternatively, a C₁ to C₂₀ hydrocarbyl group. In some embodiments, the hydrocarbyl group R can be a methyl group (—CH₃), an ethyl group (—CH₂CH₃), an isopropyl group (—CH(CH₃)₂), a tert-butyl group (—C(CH₃)₃), a phenyl group (—C₆H₅), a 2-methyl-2-propene group (—CH═C(CH₃)₂), or a 2,2-diphenylethene group (—CH═C(C₆H₅)₂). In other embodiments R can be a tert-butyl group (—C(CH₃)₃), a phenyl group (—C₆H₅), a 2-methyl-2-propene group (—CH═C(CH₃)₂), or a 2,2-diphenylethene group (—CH═C(C₆H₅)₂); alternatively, a tert-butyl group (—C(CH₃)₃) or a phenyl group (—C₆H₅); alternatively, hydrogen; alternatively, a tert-butyl group (—C(CH₃)₃); alternatively, a phenyl group (—C₆H₅); alternatively, a 2-methyl-2-propene group (—CH═C(CH₃)₂); or alternatively, a 2,2-diphenylethene group (—H═C(C₆H₅)₂).

In an embodiment, the substituted aromatic ring, Ar, of the molybdenum carbene metathesis catalyst having the structure Mo(═CHR)(NAr)(OR′)₂ can be a C₆ to C₃₀ aromatic group; alternatively, a C₆ to C₂₀ aromatic group. In some embodiments, the substituted aromatic ring, Ar, is a C₆ to C₂₀ hydrocarbyl group. In an embodiment, each substituent of the substituted aromatic ring, Ar, of the molybdenum carbene metathesis catalyst having the structure Mo(═CHR)(NAr)(OR′)₂ can independently be a C₁ to C₁₀ hydrocarbyl group; or alternatively, a C₁ to C₅ hydrocarbyl group. In some embodiments, the substituted aromatic ring, Ar, of the molybdenum carbene metathesis catalyst having the structure Mo(═CHR)(NAr)(OR′)₂ can be a 2-substituted phenyl group, a 2,6-disubstituted phenyl group, or alternatively, a 2,4,6-trisubstituted phenyl group. In an embodiment, each substituent of the substituted aromatic ring can independently be a methyl group (—CH₃), an ethyl group (—CH₂CH₃), an isopropyl group (—CH(CH₃)₂), a tert-butyl group (—C(CH₃)₃), or a neopentyl group (—CH₂C(CH₃)₃); alternatively; a methyl group (—CH₃), an isopropyl group (—CH(CH₃)₂), or a tert-butyl group (—C(CH₃)₃); alternatively a methyl group (—CH₃) or an isopropyl group (—CH(CH₃)₂). In some embodiments, each substituent of the substituted aromatic ring can independently be a methyl group (—CH₃); alternatively, an isopropyl group (—CH(CH₃)₂); or alternatively, a tert-butyl group (—C(CH₃)₃). In some non-limiting embodiments, the substituted aromatic ring, Ar, of the molybdenum carbene metathesis catalyst having the structure Mo(═CHR)(NAr)(OR′)₂ can be a 2-tert-butylphenyl group, a 2,6-dimethylphenyl group, a 2,6-diisopropylphenyl group, or a 2,4,6-trimethyl phenyl group; alternatively, a 2-tert-butylphenyl group; alternatively, a 2,6-dimethylphenyl group; alternatively, a 2,6-diisopropylphenyl group; or alternatively, a 2,4,6-trimethyl phenyl group.

In an embodiment, each R′ of the molybdenum carbene metathesis catalyst having the structure Mo(═CHR)(NAr)(OR′)₂ can independently be a C₁ to C₁₀ organic group; or alternatively a C₁ to C₅ organic group. In some embodiments, the C₁ to C₁₀ or C₁ to C₅ organic group can be a hydrocarbylhalyl group (a group consisting of hydrogen, carbon, and halogen atoms); alternatively, a hydrocarbylfluoryl group (a group consisting of hydrogen, carbon, and fluorine atoms); or alternatively, a hydrocarbyl group. In an embodiment, the halogen atoms of the hydrocarbylhalyl group can be fluorine, chlorine, bromine, iodine or combinations thereof; alternatively fluorine; alternatively, chlorine; alternatively, bromine; or alternatively, iodine. In some embodiments, each R′ of the molybdenum carbene metathesis catalyst having the structure Mo(═CHR)(NAr)(OR′)₂ can independently be tert-butyl group (—C(CH₃)₃), or a hexafluoro-tert-butyl group (—C(CF₃)₂(CH₃))group. In other embodiments, (OR′)₂ can represent a single organic group wherein the two R′ groups attached to the oxygen atoms are connected via a bond between any divalent, trivalent, or tetravalent atom with in the R′ groups. In further embodiments, (OR′)₂ can represent a single organic group wherein the two R′ groups attached to the oxygen atoms are connected via a carbon-carbon bond between any carbon atom of the two R′ groups.

In an embodiment, the molybdenum carbene metathesis catalyst can be Mo(═CH—C(CH₃)₃)(N-2,6-diisopropylphenyl)(OC(CH₃)₃), Mo(═CH—C(CH₃)₂(C₆H₅))(N-2,6-diisopropylphenyl)(OC(CH₃)₃), Mo(═CH—C(CH₃)₃)(N-2,6-diisopropylphenyl)-(OC(CH₃)(CF₃)₂), or Mo(═CH—C(CH₃)₂(C₆H₅))(N-2,6-diisopropylphenyl)(OC(CH₃)(CF₃In other embodiments, the molybdenum metathesis catalyst can be Mo(═CH—C(CH₃)₃)(N-2,6-diisopropylphenyl)(OC(CH₃)₃); alternatively, Mo(═CH—C(CH₃)₂(C₆H₅))(N-2,6-diisopropyl-phenyl)(OC(CH₃)₃); alternatively, Mo(═CH—C(CH₃)₃)(N-2,6-diisopropylphenyl)-(OC(CH₃)(CF₃)₂); or alternatively, Mo(═CH—C(CH₃)₂(C₆H₅))(N-2,6-diisopropylphenyl)-(OC(CH₃)(CF₃)₂).

In some embodiments, the metal carbene metathesis catalyst can be tethered to a support. The metal carbene metathesis catalyst can be tethered to the support via any of the ligands which do not contain the metal-carbon double bond. In an embodiment the metal carbene catalyst support may be a polymer.

Minimally, the catalyst composition comprises the metathesis catalyst (and/or the metathesis catalyst system components). In an embodiment, the catalyst composition may consist essentially of the metathesis catalyst (or the metathesis catalyst system components). In an embodiment, the catalyst composition comprising the metathesis catalyst can further comprise a solvent or diluent. In some embodiments, the catalyst composition comprising the metathesis catalyst consists essentially of the metathesis catalyst (or the metathesis catalyst system components); or alternatively, consists essentially of the metathesis catalyst (or the metathesis catalyst system components) and a solvent or diluent. Solvents or diluents which may be utilized in the catalyst composition comprising the metathesis catalyst are described herein. In other embodiments, the catalyst composition comprising the metathesis catalyst (or the metathesis catalyst system components) is substantially devoid of solvent or diluent.

Generally, the metathesis reaction may be conducted under metathesis reaction conditions which can provide a wax metathesis product having the desired constrained carbon number distribution and/or narrow melting range. Metathesis reaction conditions may include the molar ratio of metathesis catalyst moieties to moles of olefin feedstock, the temperature of the metathesis reaction, the duration of the metathesis reaction, the molar conversion of the olefin feedstock to a wax metathesis product(s), and the presence or absence of a solvent or diluent, among others. The metathesis reaction conditions are independently described herein and may be used in the combination(s) necessary to produce a wax metathesis product having a desired constrained carbon number distribution and/or narrow melting range.

In an embodiment, the molar ratio of metathesis catalyst moieties to moles of olefin feedstock can range from 1:20 to 1:1×10⁷. In some embodiments, the molar ratio of metathesis catalyst moieties to moles of olefin feedstock can range from 1:50 to 1:5×10⁶; alternatively, from 1:1×10² to 1:1:1×10⁶; alternatively, from 1:5×10² to 1:1:5×10⁵; alternatively, from 1:1×10³ to 1:1:1×10⁵;or alternatively, from 1:5×10³ to 1:1:5×10⁴.

Generally, the metathesis reaction is performed at a temperature which maintains the metathesis reaction solution in a processable state. Applicable temperatures for the metathesis reaction are described herein. In an embodiment, a solvent or diluent may be used to maintain the reaction solution in a processable state. Depending upon the particular olefin feedstock, wax metathesis product, and other factors (e.g. the presence of absence or a solvent or diluent) a metathesis reaction temperature greater than the melting point of the olefin feedstock may be required in order to maintain the metathesis reaction solution in a processable state. The term “processable state” refers to a solution which can be stirred, pumped, and/or is sufficiently fluid to flow through a column. Consequently, a metathesis reaction solution in a “processable state” does not necessarily refer to metathesis reaction solution wherein all materials are in the liquid and/or gaseous state. For example, the processable solution may comprise solid (non-liquid or undissolved) wax particles which do not prevent the ability to stir and/or pump the metathesis reaction solution or impede the metathesis reaction solution flow through a column.

In an embodiment, the metathesis reaction temperature may be any temperature which maintains the metathesis reaction solution in a processable state. In some embodiments, the metathesis reaction temperature may be any temperature greater than the melting point of the olefin feedstock. In other embodiments, the metathesis reaction temperature may a temperature less that the melting point of the olefin feedstock when a solvent or diluent is utilized to maintain the metathesis reaction solution in a processable state. In an embodiment, the metathesis reaction temperature, regardless of the presence or absence of a solvent or diluent, may range from the melting point of the olefin feedstock to 250° C.; or alternatively, ranging from the melting point of the olefin feedstock to 150° C. In some non-limiting embodiments, the metathesis reaction temperature can be greater than 50° C.; alternatively, greater than 60° C.; or alternatively, greater than 70° C. In other embodiments, the metathesis reaction temperature, regardless of the presence or absence of a solvent or diluent, can range from 30° C. to 150° C.; alternatively, range from 50° C. to 150° C.; alternatively, range from 60° C. to 120° C.; alternatively, range from 70° C. to 120° C.; or alternatively, range from 70° C. to 110° C. In further embodiments, the metathesis reaction temperature, regardless of the presence or absence of a solvent or diluent, may range from 15° C. to 70° C.; alternatively, range from 15° C. to 60° C.; alternatively, range from 15° C. to 50° C.; or alternatively, range from 15° C. to 40° C. In yet other embodiments, the metathesis reaction temperature may range from 80° C. to 250° C.; alternatively, range from 100° C. to 200° C.; alternatively, range from 110° C. to 180° C.; or alternatively, range from 120° C. to 160° C. One of ordinary skill in the art will recognize that to maintain the metathesis catalyst in a processable state, a solvent or diluent will need to be utilized when a metathesis reaction temperature is less than the melting point of the olefin feedstock.

Generally, the metathesis reaction may be carried out for a duration which can produce a wax metathesis product having the desired constrained carbon number distribution and/or narrow melting range. The metathesis reaction duration which may be utilized to produce a wax metathesis product having the desired constrained carbon number distribution and/or narrow melting range may be dependent upon the identity of the metathesis catalyst used, the molar ratio of metathesis catalyst moieties to moles of olefin feedstock, the olefin conversion to wax metathesis products, and the metathesis reaction temperature, among other metathesis reaction parameters (e.g. the identity of the olefin feedstock). In an embodiment, the duration of the metathesis reaction can range from 1 minute to 48 hours. In some embodiments, the duration of the metathesis reaction can range from 10 minutes to 30 hours. In other embodiments, the duration of the metathesis reaction can range from 15 minutes to 24 hours; alternatively, 15 minutes to 12 hours; alternatively, 15 minutes to 6 hours; or alternatively, 15 minutes to 4 hours.

Generally, the metathesis reaction may be carried out to achieve a molar conversion of olefin feedstock to wax metathesis product, ((Moles Wax Metathesis product*2)/Moles Olefin Feedstock)*100, that can produce a wax metathesis product having the desired constrained carbon number distribution and/or narrow melting range. The conversion to wax metathesis product having the desired constrained carbon number distribution and/or narrow melting range may be dependent upon the identity of the metathesis catalyst used, the molar ratio of metathesis catalyst moieties to moles of olefin feedstock, and the metathesis reaction temperature, among other metathesis reaction parameters (e.g. the identity of the olefin feedstock). In an embodiment, the molar conversion of the olefin feedstock to wax metathesis product can be greater than 30 mole percent; alternatively, greater than 50 mole percent; alternatively, greater than 60 mole percent; alternatively, greater than 70 mole percent; or alternatively, greater than 80 mole percent. In some embodiments, the molar conversion of the olefin feedstock to wax metathesis product can range from 30 mole percent to 100 mole percent; alternatively, from 50 mole percent to 100 mole percent; alternatively, from 60 mole percent to 98 mole percent; alternatively, from 70 mole percent to 95 mole percent; or alternatively, from 80 mole percent to 90 mole percent.

In an embodiment, the metathesis reaction is performed under conditions which can remove a portion or all of the light metathesis product(s) (e.g. ethylene and propylene) from the metathesis reaction solution. In some embodiments, the metathesis reaction is performed under conditions wherein some or a portion of the light metathesis products are a gas and can degas from the metathesis reaction solution. In an embodiment, the light metathesis product(s) can be removed by performing the metathesis reaction at ambient pressure (e.g. atmospheric pressure); or alternatively, at sub-ambient pressure (e.g. pressure less than 101 kPa). In some embodiments, the light metathesis product(s) can be removed by performing the metathesis reaction with an inert gas sweep (passing an inert gas through the metathesis reaction solution and/or headspace). In other embodiments, the light metathesis product(s) can be removed by performing the metathesis reaction at about ambient pressure with an inert gas sweep; or alternatively, at sub-ambient pressure with an inert gas sweep.

In an embodiment, the metathesis reaction can be performed in the presence of a solvent or diluent. In some embodiments, the metathesis reaction is performed in the substantial absence of a solvent or diluent. Generally, the solvent or diluent which may be utilized for the metathesis reaction can be any solvent that is compatible with the metathesis catalyst utilized. Solvents or diluents which may be utilized for the metathesis reaction are described herein. Persons with skill in the art will recognize which solvent or diluent classes and/or specific solvents or diluents are compatible with a particular metathesis catalyst class or specific metathesis catalyst.

In an embodiment, the feedstock composition comprising the olefin feedstock, the catalyst composition comprising the metathesis catalyst, and/or the metathesis reaction solution may further comprise a solvent or diluent. In some embodiments, the solvent or diluent used for the feedstock composition comprising the olefin feedstock, the catalyst composition comprising the metathesis catalyst, and/or the reaction solution can be the same; or alternatively, the solvent or diluent may be different for one or more of the compositions.

The solvent or diluent utilized for the feedstock composition comprising the olefin feedstock, the catalyst composition comprising the metathesis catalyst, and/or the reaction solution can comprise, or consist essentially of, a hydrocarbon, a halogenated hydrocarbon, an ether, or combinations thereof. In some embodiments, the solvent or diluent, can comprise, or consist essentially of, a hydrocarbon; alternatively, a halogenated hydrocarbon; or alternatively, an ether. In some embodiments, the hydrocarbon solvent or diluent can be a saturated hydrocarbon; or alternatively, an aromatic hydrocarbon.

In an embodiment, the solvent or diluent can comprise, or consist essentially of, a C₄ to C₂₀ hydrocarbon; or alternatively, a C₅ to C₁₀ hydrocarbon. In some embodiments, the solvent or diluent can comprise, or consist essentially of, a C₄ to C₂₀ saturated hydrocarbon; alternatively, a C₅ to C₁₀ saturated hydrocarbon. In some embodiments, the solvent or diluent can comprise, or consist essentially of, a C₆ to C₂₀ aromatic hydrocarbon; or alternatively, C₆ to C₁₀ aromatic hydrocarbon. In some embodiments, the solvent or diluent can comprise, or consist essentially of, a C₁ to C₁₅ halogenated hydrocarbon; alternatively, C₁ to C₁₀ halogenated hydrocarbon; or alternatively, C₁ to C₅ halogenated hydrocarbon. In some embodiments, the solvent or diluent can comprise, or consist essentially of, a C₂ to C₁₀ ether; or alternatively, a C₂ to C₅ ether.

Suitable saturated hydrocarbon solvent(s) or diluent(s) can include butane, isobutane, pentane, n-hexane, hexanes, cyclohexane, n-heptane, n-octane, or mixtures thereof; or alternatively, n-hexane, hexanes, cyclohexane, n-heptane, n-octane, or mixtures thereof. Suitable aromatic hydrocarbon solvent(s) or diluent(s) can include benzene, toluene, mixed xylenes, ortho-xylene, meta-xylene, para-xylene, ethylbenzene, or mixtures thereof. Suitable halogenated solvent(s) or diluent(s) can include carbon tetrachloride, chloroform, methylene chloride, dichloroethane, trichloroethane, chlorobenzene, or dichlorobenzene, or mixtures thereof. Suitable ether solvent(s) or diluent(s) can include dimethyl ether, diethyl ether, methyl ethyl ether, monoethers or diethers of glycols (e.g. dimethyl glycol ether), furans, substituted furans, dihydrofuran, substituted dihydrofurans, tetrahydrofuran (THF), substituted tetrahydrofurans, tetrahydropyrans, substituted tetrahydropyrans, 1,3-dioxanes, substituted 1,3-dioxanes, 1,4-dioxanes, substituted 1,4-dioxanes, or mixtures thereof. In an embodiment, each substituent of a substituted furan, substituted dihydrofuran, substituted tetrahydrofuran, substituted tetrahydropyran, substituted 1,3-dioxane, or substituted 1,4-dioxane, can be a C₁ to C₅ alkyl group.

The wax metathesis product produced by the methods described herein can be described using either singly, or in any combination, features including the type of molecules (e.g. organic, hydrocarbon, linear, branched, aliphatic, aromatic, acyclic, cyclic, mono-olefinic, among others described herein), the number of carbon atoms present in the wax metathesis product molecules, the average molecular weight of the wax metathesis product molecules, features of the constrained carbon number distribution of the wax metathesis product molecules, and/or melting features of the wax metathesis product. These wax metathesis product features are independently described herein and may be used in any combination to describe the wax metathesis product and/or the wax metathesis product molecules.

The constrained carbon number distribution of the wax metathesis product and/or wax metathesis product molecules can be described using, either singly or in any combination, the weight percentage of wax metathesis product having a carbon number ranging from 2FC−2 to 2MC−2, the weight percentage of wax metathesis product molecules having a molecular weight less than 2FC−2, the weight percentage of wax metathesis product molecules having a molecular weight within a specified number of grams/mole of the average molecular weight of the wax metathesis product, and/or the carbon number span of the wax metathesis product molecules, among other parameters described herein. In some non-limiting embodiments, the wax metathesis product produced by the methods described herein can be described using any average molecular weight of the wax metathesis product molecules described herein in combination with any feature describing the constrained carbon number of the wax metathesis product and/or wax metathesis product molecules described herein. The melting features which can be utilized to describe the wax metathesis product and/or wax metathesis product molecules can include the DSC melting range of the wax metathesis product or a combination of the DSC melting point and the DSC melting range of the wax metathesis product. Additionally, in a non-limiting embodiment, the wax metathesis product produced by the methods described herein can be described using any combination of the constrained carbon number criteria described herein and the narrow melting criteria described herein.

In an embodiment, the wax metathesis product may comprise, or consist essentially of, aliphatic olefins, aromatic olefins, or combinations thereof; alternatively, aliphatic olefins; or alternatively, aromatic olefins. In an embodiment, the wax metathesis product (whether aliphatic or aromatic) can comprise, or consist essentially of, linear olefins, branched olefins, or combinations thereof; alternatively, linear olefins; or alternatively, branched olefins. In other embodiments, the wax metathesis product (whether aliphatic or aromatic, linear or branched, or combinations thereof) may comprise, or consist essentially of, acyclic olefins, cyclic olefins or combinations thereof; alternatively, acyclic olefins; or alternatively, cyclic olefins. In an embodiment, the wax metathesis product (whether aliphatic or aromatic, linear or branched, cyclic or acyclic, or combinations thereof) may comprise, or consists essentially of, hydrocarbons. In some embodiments, the wax metathesis product (whether aliphatic or aromatic, linear or branched, cyclic or acyclic, hydrocarbon or combinations thereof) may comprise, or consist essentially of, mono-olefins. In other embodiments, the wax metathesis product (whether aliphatic or aromatic, linear or branched, cyclic or acyclic, or combinations thereof) may comprise, or consist essentially of, hydrocarbon mono-olefins. In some other embodiments, the wax metathesis product (whether aliphatic or aromatic, linear or branched, cyclic or acyclic, hydrocarbon, mono-olefinic, or combinations thereof) can comprise, or consist essentially of, internal olefinic wax molecules. In some other embodiments, the wax metathesis product (whether aliphatic or aromatic, cyclic or acyclic, hydrocarbon, mono-olefinic, or combinations thereof) can comprise, or consist essentially of, linear internal olefinic wax molecules. In some other embodiments, the wax metathesis product (whether aliphatic or aromatic, linear or branched, cyclic or acyclic, hydrocarbon, mono-olefinic, or combinations thereof) can comprise, or consist essentially of, internal disubstituted olefinic wax molecules. In some other embodiments, the wax metathesis product (whether aliphatic or aromatic, linear or branched, cyclic or acyclic, hydrocarbon, mono-olefinic, internal, linear internal, internal disubstituted, or combinations thereof) can comprise, or consist essentially of, olefinic wax molecules wherein the carbon-carbon double bond is at least 6, 8, 10, or 12 carbon atoms from each of the two terminal carbon atoms of the longest continuous chain of carbon atoms. In other embodiments, the wax metathesis product (whether aliphatic or aromatic, linear or branched, cyclic or acyclic, hydrocarbon, mono-olefinic, internal, linear internal, internal disubstituted, or combinations thereof) can comprise, or consist essentially of, olefins wherein the two terminal carbon atoms of the longest continuous chain of carbon atoms and the carbon-carbon double bond are separated by at least 6 carbon atoms; alternatively, 8 carbon atoms; alternatively, 10 carbon atoms; or alternatively, 12 carbon atoms. In further embodiments, the wax metathesis product (whether aliphatic or aromatic, linear or branched, cyclic or acyclic, mono-olefinic, internal, linear internal, internal disubstituted, or combinations thereof) can comprise, or consist essentially of, olefinic hydrocarbon wax molecules wherein the carbon-carbon double bond is at least 6, 8, 10, or 12 carbon atoms from each of the two terminal carbon atoms of the longest continuous chain of carbon atoms. In other embodiments, the wax metathesis product (whether aliphatic or aromatic, linear or branched, cyclic or acyclic, mono-olefinic, internal, linear internal, internal disubstituted, or combinations thereof) can comprise, or consist essentially of, hydrocarbon olefins wherein the two terminal carbon atoms of the longest continuous chain of carbon atoms and the carbon-carbon double bond is separated by at least 6 carbon atoms; alternatively, 8 carbon atoms; alternatively, 10 carbon atoms; or alternatively, 12 carbon atoms. In yet further embodiments, the wax metathesis product (whether aliphatic or aromatic, linear or branched, cyclic or acyclic, mono-olefinic, internal, linear internal, internal disubstituted, or combinations thereof) can comprise, or consist essentially of, olefinic hydrocarbon wax molecules having an even and odd number of carbon atoms; alternatively, an odd number of carbon atoms; or alternatively, an even number of carbon atoms.

Generally, an unhydrogenated wax metathesis product will share the features of the olefin feedstock which is used to produce the wax metathesis product. For example, when the olefin feedstock comprises, or consists essentially of, branched olefins, the wax metathesis product may comprise, or consist essentially of, branched molecules; alternatively, when an olefin feedstock consists essentially of hydrocarbon olefins the wax metathesis product may consist essentially of hydrocarbon olefin molecules. In other embodiments, when the olefin feedstock consists essentially of a non-branched (linear) olefin feedstock described herein, the wax metathesis product may consist essentially of linear molecules (a linear wax metathesis product). In other embodiments, when the olefin feedstock consists essentially of any linear hydrocarbon olefin feedstock described herein (e.g. generic linear hydrocarbon olefins or normal alpha olefins, among others), the wax metathesis product, may consist essentially of linear hydrocarbon molecules (a linear hydrocarbon wax metathesis product).

In an embodiment, the wax metathesis product may comprise, or consist essentially of, wax metathesis product molecules having greater than 30 carbon atoms; alternatively greater than 34 carbon atoms; alternatively greater than 40 carbon atoms; alternatively greater than 45 carbon atoms; or alternatively greater than 50 carbon atoms. In some embodiments, the wax metathesis product may comprise, or consist essentially of, wax metathesis product molecules having from 30 to 100 carbon atoms; alternatively, from 30 to 80 carbon atoms; alternatively, from 30 to 60 carbon atoms; alternatively, from 30 to 50 carbon atoms; alternatively, from 30 to 40 carbon atoms; alternatively, from 34 to 80 carbon atoms; alternatively, from 34 to 60 carbon atoms; alternatively, from 34 to 50 carbon atoms; alternatively, from 40 to 80 carbon atoms; alternatively, from 40 to 70 carbon atoms; alternatively, from 40 to 60 carbon atoms; alternatively, from 45 to 60 carbon atoms; or alternatively, from 50 to 80 carbon atoms.

In an embodiment, the wax metathesis product having a constrained carbon number distribution may have a carbon number span less than 15. In some embodiments, the wax metathesis product having a constrained carbon number distribution may have a carbon number span less than 13; alternatively, less than 11; alternatively, less than 9; or alternatively, less than 7. In other embodiments, the wax metathesis product having a constrained carbon number distribution may have a carbon number span greater than 1 and less than 15; alternatively, greater than 1 and less than 13; alternatively, greater than 1 and less than 11; alternatively, greater than 1 and less than 9; or alternatively, greater than 1 and less than 7. In other embodiments, the wax metathesis product having a constrained carbon number distribution may have a carbon number span greater than 2 and less than 15; alternatively, greater than 2 and less than 13; alternatively, greater than 2 and less than 11; alternatively, greater than 2 and less than 9; or alternatively, greater than 2 and less than 7. In yet other embodiments, the wax metathesis product having a constrained carbon number distribution may have a carbon number span greater than 3 and less than 15; alternatively, greater than 3 and less than 13; alternatively, greater than 3 and less than 11; alternatively, greater than 3 and less than 9; or alternatively, greater than 3 and less than 7. In other embodiments, the wax metathesis product having a constrained carbon number distribution may have a carbon number span greater than 4 and less than 15; alternatively greater than 4 and less than 13; alternatively, greater than 4 and less than 11; or alternatively, greater than 4 and less than 9. In further embodiments, the wax metathesis product having a constrained carbon number distribution may have a carbon number span greater than 5 and less than 15; alternatively, greater than 5 and less than 13; alternatively, greater than 5 and less than 11; or alternatively, greater than 5 and less than 9.

In an embodiment, the wax metathesis product having a constrained carbon number distribution may have a carbon number distribution wherein greater than 70 weight percent of the wax metathesis product has a carbon number ranging from 2FC−2 to 2MC−2, wherein FC equals the carbon number of the olefin feedstock having the fewest carbon atoms present in an amount of at least 3 weight percent and MC equals the carbon number of the olefin feedstock having the greatest number of carbon atoms present in an amount of at least 3 weight percent. In some embodiments, the wax metathesis product having a constrained carbon number distribution may have a carbon number distribution wherein greater than 75 weight percent of the wax metathesis product has a carbon number ranging from 2FC−2 to 2MC−2; alternatively, wherein greater than 80 weight percent of the wax metathesis product has a carbon number ranging from 2FC−2 to 2MC−2; alternatively, wherein greater than 85 weight percent of the wax metathesis product has a carbon number ranging from 2FC−2 to 2MC−2; alternatively, wherein greater than 90 weight percent of the wax metathesis product has a carbon number ranging from 2FC−2 to 2MC−2; or alternatively, wherein greater than 95 weight percent of the wax metathesis product has a carbon number ranging from 2FC−2 to 2MC−2.

In an embodiment, the wax metathesis product having a constrained carbon number distribution may have a carbon number distribution wherein less than 30 weight percent of wax metathesis product has a carbon number less than 2FC−2. In some embodiments, the wax metathesis product having a constrained carbon number distribution may have a carbon number distribution wherein less than 25 weight percent of the wax metathesis product has a carbon number less than 2FC−2; alternatively, less than 20 weight percent of the wax metathesis product has a carbon number less than 2FC−2; alternatively, less than 15 weight percent of the wax metathesis product has a carbon number less than 2FC−2; or alternatively, less than 10 weight percent of the wax metathesis product has a carbon number less than 2FC−2.

In an embodiment, the wax metathesis product having a constrained carbon number distribution may have a carbon number distribution wherein greater than 70 weight percent of the wax metathesis molecules have a molecular weight within 42 grams/mole of the average molecular weight of the wax metathesis product molecules; alternatively, within 35 grams/mole of the average molecular weight of the wax metathesis product molecules; alternatively, within 28 grams/mole of the average molecular weight of the wax metathesis product molecules; or alternatively, within 21 grams/mole of the average molecular weight of the wax metathesis product molecules. In some embodiments, the wax metathesis product having a constrained carbon number distribution may have a carbon number distribution wherein greater than 80 weight percent of the wax metathesis product molecules have a molecular weight within 49 grams/mole of the average molecular weight of the wax metathesis product molecules; alternatively, within 42 grams/mole of the average molecular weight of the wax metathesis product molecules; alternatively, within 35 grams/mole of the average molecular weight of the wax metathesis product molecules; or alternatively, within 28 grams/mole of the average molecular weight of the wax metathesis product molecules. In other embodiments, the wax metathesis product having a constrained carbon number distribution may have a carbon number distribution wherein greater than 85 weight percent of the wax metathesis product molecules have a molecular weight within 56 grams/mole of the average molecular weight of the wax metathesis product molecules; alternatively, within 49 grams/mole of the average molecular weight of the wax metathesis product molecules; alternatively, within 42 grams/mole of the average molecular weight of the wax metathesis product molecules; or alternatively, within 35 grams/mole of the average molecular weight of the wax metathesis product molecules. In further embodiments, the wax metathesis product having a constrained carbon number distribution may have a carbon number distribution wherein greater than 90 weight percent of the wax metathesis product molecules have a molecular weight within 63 grams/mole of average molecular weight of the wax metathesis product molecules; alternatively, within 56 grams/mole of the average molecular weight of the wax metathesis product molecules; alternatively, within 49 grams/mole of the average molecular weight of the wax metathesis product molecules; or alternatively, within 42 grams/mole of the average molecular weight of the wax metathesis product molecules. In yet other embodiments, the wax metathesis product having a constrained carbon number distribution may have a carbon number distribution wherein greater than 95 weight percent of the wax metathesis product molecules have a molecular weight within 63 grams/mole of the average molecular weight of the wax metathesis product molecules; alternatively, within 56 grams/mole of the average molecular weight of the wax metathesis product molecules; alternatively, within 49 grams/mole of the average molecular weight of the wax metathesis product molecules; or alternatively, within 42 grams/mole of the average molecular weight of the wax metathesis product molecules.

In an embodiment, the wax metathesis product molecules of the wax metathesis product having a constrained carbon number distribution may have an average molecular weight greater than 400 grams/mole; alternatively, greater than 450 grams/mole; alternatively, greater than 500 grams/mole; alternatively, greater than 600 grams/mole; or alternatively, greater than 800 grams/mole. In some embodiments, the wax metathesis product molecules of the wax metathesis product having a constrained carbon number distribution may have an average molecular weight ranging from 400 to 5000 grams/mole; alternatively, ranging from 450 to 2500 grams/mole; alternatively, ranging from 450 to 1500 grams/mole; alternatively, ranging from 400 to 1200 grams/mole; alternatively, ranging from 450 to 1000 grams/mole; alternatively, ranging from 450 to 600 grams/mole; alternatively, ranging from 500 to 1000 grams/mole; alternatively, ranging from 500 to 700 grams/mole; alternatively, ranging from 600 to 1000 grams/mole alternatively, ranging from 600 to 850 grams/mole; or alternatively, ranging from 750 to 1200 grams/mole.

In an embodiment, the wax metathesis product having a narrow DSC melting range, and/or constrained carbon number distribution may have a DSC melting range less than 20° C. In some embodiments the wax metathesis product having a narrow DSC melting range and/or constrained carbon number distribution may have a DSC melting range less than 15° C.; alternatively, less than 13° C.; alternatively, less than 12° C.; alternatively, less than 11° C.; or alternatively, less than 10° C. In some embodiments, the wax metathesis product having a narrow DSC melting range and/or constrained carbon number distribution may have a DSC melting range greater than 3° C. and less than 20° C.; alternatively, greater than 3° C. and less than 15° C.; alternatively, greater than 3° C. and less than 13° C.; alternatively, greater than 3° C. and less than 12° C.; alternatively, greater than 3° C. and less than 11; alternatively, greater than 3° C. and less than 10° C., alternatively, greater than 5° C. and less than 20° C.; alternatively, greater than 5° C. and less than 15° C.; alternatively, greater than 5° C. and less than 13° C.; alternatively, greater than 5° C. and less than 12° C.; alternatively, greater than 5° C. and less than 11; or alternatively, greater than 5° C. and less than 10° C.

In an embodiment, the wax metathesis product having any DSC melting range described herein, and/or constrained carbon number distribution described herein, may have a DSC melting point greater than 40° C.; alternatively greater than 50° C.; alternatively greater than 60° C.; or alternatively, greater than 65° C. In some embodiments, the wax metathesis product having any DSC melting range described herein, and/or constrained carbon number constrained carbon number distribution described herein, may have a DSC melting point ranging from 40° C. to 200° C.; alternatively, ranging from 40° C. to 150° C.; alternatively, ranging from 40° C. to 100° C.; alternatively, ranging from 50° C. to 150° C.; alternatively, ranging from 50° C. to 130° C.; alternatively, ranging from 50° C. to 100° C.; alternatively, ranging from 60° C. to 150° C.; alternatively, ranging from 60° C. to 130° C.; alternatively, ranging from 60° C. to 100° C.; alternatively, ranging from 60° C. to 80° C.; alternatively, ranging from 80° C. to 150° C.; alternatively, ranging from 80° C. to 130° C.; or alternatively, ranging from 80° C. to 100° C.

In an embodiment, the method(s) for producing a wax metathesis product having a constrained carbon number distribution and/or a narrow melting range may further comprise separating the wax metathesis product from the metathesis catalyst, the unconverted olefin feedstock, the non-wax metathesis product olefin, any component of the composition comprising the metathesis catalyst composition (e.g. solvent or diluent), any component of the feedstock composition comprising the olefin feedstock (e.g. solvent or diluent), and/or any component of the metathesis reaction solution (e.g. solvent of diluent). In some embodiments, the method(s) for producing a wax metathesis product having a constrained carbon number distribution and/or a narrow melting range may further comprise separating the wax metathesis product from the metathesis catalyst (or metathesis catalyst components). In other embodiments, the method(s) for producing the wax metathesis product having a constrained carbon number distribution and/or a narrow melting range may further comprise separating the wax metathesis product from the unconverted olefin feedstock. In other embodiments, the method(s) for producing the wax metathesis product having a constrained carbon number distribution and/or a narrow melting range may further comprise separating the wax metathesis product from the non-wax metathesis product olefin. In further embodiments, the method(s) for producing the wax metathesis product having a constrained carbon number distribution and/or a narrow melting range may comprise separating the wax metathesis product from any solvent(s) or diluent(s) utilized in the method for producing a wax metathesis product; for example any solvent(s) or diluent(s) utilized in the feedstock composition comprising the olefin feedstock, catalyst composition comprising the metathesis catalyst, and/or the metathesis reaction solution. In other embodiments, the method(s) for producing the wax metathesis product having a constrained carbon number distribution and/or a narrow melting range may comprise separating the wax metathesis product from the unconverted olefin feedstock, the non-wax metathesis product olefin and any solvent(s) or diluent(s) utilized in the method for producing a wax metathesis product. In yet other embodiments, the method(s) for producing a wax metathesis product having a constrained carbon number distribution and/or a narrow melting range may comprise separating the wax metathesis product from the metathesis catalyst, the unconverted olefin feedstock, the non-wax metathesis product olefin, and separating the wax metathesis product from any solvent(s) or diluent(s) utilized in the method for producing a wax metathesis product. In an embodiment, the separation of the wax metathesis product from the other components in the metathesis reaction solution may be performed concurrently; or alternatively, in separate steps. When the wax metathesis product is separated from the unconverted olefin feedstock, the non-wax metathesis product olefin, and the solvent(s) or diluent(s) in two or more separate steps, the separations can occur in any order. In further embodiments, the method(s) for producing a wax metathesis product having a constrained carbon number distribution and/or narrow melting range may comprise hydrogenating the wax metathesis product.

In an embodiment, the wax metathesis product can be separated from a composition comprising the metathesis catalyst (or the metathesis catalyst components) by decantation, filtration, absorption, or any combination thereof. In some embodiments, the wax metathesis product can be separated from a composition comprising the metathesis catalyst (or the metathesis catalyst components) by decantation; alternatively, filtration; or alternatively, absorption. In some embodiments, the separation of the metathesis catalyst (or the metathesis catalyst components) from the wax metathesis product may be performed by passing the composition comprising the wax metathesis product and the metathesis catalyst (or the metathesis catalyst components) through an absorbent bed. In an embodiment, multiple separation steps can be performed. In a non-limiting example, the wax metathesis product can be separated from the metathesis catalyst (or the metathesis catalyst components) by subjecting the composition comprising the wax metathesis product and metathesis catalyst (or the metathesis catalyst components) to decantation followed by passing the composition comprising the wax metathesis product and metathesis catalyst (or the metathesis catalyst components) through an absorbent bed. One of ordinary skill in the art will recognize that other method steps may precede, occur after, and/or occur between the multiple steps of separating the wax metathesis product from the metathesis catalyst (or the metathesis catalyst components). For example wax metathesis product hydrogenation, or removing the non-wax metathesis product olefin from the wax metathesis product, among other method steps may precede, occur after, and/or occur between the multiple steps of separating the wax metathesis product from the metathesis catalyst (or the metathesis catalyst components).

When the wax metathesis product is separated from the metathesis catalyst (or the metathesis catalyst components) by absorption, the absorbent can be any absorbent which can separate the metathesis catalyst from the wax metathesis product. In an embodiment, the absorbent can be silica, activated carbon, alumina, or combinations thereof. In some embodiments, the absorbent can be alumina. In embodiments wherein the absorbent is alumina, the alumina can be any alumina which can bind the metathesis catalyst (or the metathesis catalyst components) used to prepare the wax metathesis product. In an embodiment, the absorbent comprises α-alumina, β-alumina, γ-alumina or combinations thereof. In some embodiments, the absorbent is α-alumina; alternatively, β-alumina; or alternatively, γ-alumina. Generally, when the absorbent is an alumina (α-alumina, β-alumina, γ-alumina, or combinations thereof), the alumina can be acidic, neutral, or basic. In some embodiments, the alumina, whether the alumina is α-alumina, β-alumina, γ-alumina or combinations thereof, can be acidic; alternatively, neutral; or alternatively, or basic.

In an embodiment, the wax metathesis product (or hydrogenated wax metathesis product) can be separated from the unconverted olefin feedstock, non-wax metathesis product olefin, and/or any solvent(s) or diluent(s) (if present) utilized in the method by distillation, selective crystallization, size exclusion adsorption, recrystallization, or fractional crystallization. In some embodiments, the wax metathesis product (or hydrogenated wax metathesis product) is separated from the unconverted olefin feedstock, non-wax metathesis product olefin, and/or any solvent(s) or diluent(s) (if present) by distillation; alternatively, size exclusion adsorption; alternatively, recrystallization; or alternatively, fractional crystallization. In yet other embodiments, two or more separating steps may be utilized. Two non-limiting examples of using two or more separating steps to separate the wax metathesis product (or hydrogenated wax metathesis product) from the unconverted olefin feedstock, non-wax metathesis product olefin, and/or any solvent(s) or diluent(s) (if present) can include using two or more distillation steps; or alternatively using a distillation step and a selective crystallization, recrystallization, thermal fractionation, or fractional crystallization step.

Generally, the recrystallization may be performed in any solvent in which the olefin feedstock, non-wax metathesis product olefin, and/or any solvent(s) or diluent(s) are soluble. In an embodiment, the fractional crystallization step may be preformed in the presence of a solvent; or alternatively, in the substantial absence of a solvent. In some embodiments, the recrystallization, or fractional crystallization solvent is a hydrocarbon; or alternatively, a halogenated hydrocarbon. In some embodiments, the recrystallization, or fractional crystallization solvent is an olefinic hydrocarbon; or alternatively, a saturated hydrocarbon. Generally, the hydrocarbon recrystallization solvent, or fractional crystallization solvent can be a C₆ to C₁₆ hydrocarbon; or alternatively, a C₁ to C₁₀ halogenated hydrocarbon.

In an embodiment, the method(s) for producing a wax metathesis product having a constrained carbon number distribution and/or narrow melting range may further comprise hydrogenating the wax metathesis product. Generally, the hydrogenation step can utilize any hydrogenation catalyst or hydrogenation method known to those of ordinary skill in the art. For example, either a nickel or palladium based hydrogenation catalyst can be utilized to hydrogenate the wax metathesis product. Depending upon the specifics of the method and the metathesis reaction conditions (i.e. conversion of the olefin feedstock to wax metathesis product, desirability of recycling the unconverted olefin feedstock, and the compatibility of the metathesis catalyst and the hydrogenation catalyst, among other considerations), the hydrogenation can be performed at any point in the method after the production of the wax metathesis product. In an embodiment, the hydrogenation is performed after the separation of the wax metathesis product from the unconverted olefin feedstock, the non-wax metathesis product olefin, and/or any solvent(s) or diluent(s) (if present) utilized in the method for producing a wax metathesis product. In some embodiments, the wax metathesis product may be hydrogenated in the presence of the unconverted olefin feedstock and/or the non-wax metathesis product olefin. In yet other embodiments, the wax metathesis product is hydrogenated after the wax metathesis product is separated from the metathesis catalyst.

A common phenomenon in olefin metathesis is that the metathesis catalyst isomerizes the olefin feedstock and/or the metathesis product. The isomerization of the olefin feedstock and/or metathesis product leads to new olefins which further participate in the olefin metathesis reaction and results in the production of more metathesis products than would be predicted based upon the identity of the individual olefins of the olefin feedstock. For example, in the hypothetical absence of olefin isomerization, the metathesis of an olefin feedstock consisting of alpha olefins will produce ethylene and olefins having a carbon number ranging from 2FC−2 to 2MC−2. In a specific hypothetical absence of isomerization example, the self metathesis of 1-heptene would produce only ethylene and 6-dodecene. However, when the metathesis catalyst isomerizes the feedstock olefin, additional olefins will be produced and can participate in the metathesis reactions. For example, in the metathesis of 1-heptene, the olefin feedstock 1-heptene, may be isomerized to 2-heptene. The 2-heptene may then react, via metathesis, with 1-heptene, ethylene, 6-dodecene, or itself to produce propene, 2-butene, 1-hexene, 5-decene, and 5-undecene in addition to the ethylene and 6-dodecene produced from the self metathesis of 1-heptene. To complicate matters further, the 2-heptene and/or the 1-heptene may be isomerized to 3-heptene which will react, via metathesis reactions, with each of the previously mentioned compounds to produce 1-butene, 1-pentene, 2-pentene, 2-hexene, 3-hexene, 3-octene, 4-octene, 3-nonene, 4-nonene, and 4-decene in addition to the metathesis products from the self metathesis and cross metathesis of 1-heptene and 2-heptene. Additionally, the metathesis products can be isomerized by the metathesis product and may participate in further metathesis and/or isomerization reactions with the olefin feedstock, the isomerized olefin feedstock, the metathesis product, and/or the isomerized metathesis product to produce even more compounds. For example, the 6-dodecene produced by the metathesis of 1-heptene can isomerize to 5-dodecene and can react with 1-heptene, 6-dodecene, and 5-dodecene (not to mention the other olefins in the metathesis reaction solution) to produce 1-hexene, 1-octene, 5-decene, 5-undecene, 6-tridecene, and 7-tetradecene, among other metathesis products. Further, isomerization and metathesis reaction can occur producing additional metathesis products. Consequently, isomerization of the olefin feedstock and the metathesis products result in the broadening of the carbon number distribution of the metathesis products having a carbon number less than the olefin feedstock and the broadening of the carbon number distribution of the metathesis products having a carbon number greater than the carbon number of the olefin feedstock beyond that predicted in the absence of olefin isomerization. Furthermore, because olefin isomerization rate of the metathesis catalyst increases with increasing temperature and/or increase with increasing reaction time, the carbon number broadening of the metathesis products having carbon numbers less than and/or greater than the olefin feedstock increases with increasing reaction time and/or increasing reaction temperature.

When dealing with long-chained feedstocks, (e.g. those having greater than 15 carbon atoms) and/or olefin feedstocks having a mixture of olefins having different carbon numbers (and/or structures), the situation is even more complicated because, as the carbon chain lengths increase, the number of different olefins produced by olefin isomerization of the olefin feedstock and/or the wax metathesis product, which subsequently participate in the metathesis reactions, increase. Additionally, as the carbon number of the olefin feedstock increases, the olefin feedstocks and/or metathesis product become waxes at ambient conditions (20° C. -25° C. and 101 kPa) and the metathesis reaction temperature may need to be increased to maintain the metathesis reaction solution in a processable state. Unfortunately, olefin isomerization activity of metathesis catalysts and the rate of olefin isomerization increases with increasing temperature; this is even true of metathesis catalysts which reportedly have little or no isomerization activity at ambient temperature. Consequently, the production of a wax metathesis product having a desired constrained carbon number distribution and/or narrow melting range is not a trivial task as a desired wax metathesis product having a constrained carbon number distribution and/or narrow melting range may not be obtained from every conceivable combination of olefin feedstock, metathesis catalyst, and metathesis reaction conditions. For example, metal oxide and metal halide systems are particularly good at isomerizing olefins and may not suitable for metathesizing a particular olefin under all conceivable combinations of metathesis reaction conditions to produce a wax metathesis product having a desired constrained carbon number distribution and/or narrow melting range. Additionally, metal carbene metathesis catalyst can also isomerize olefin and may not suitable for metathesizing a particular olefin under all conceivable combinations of metathesis reaction conditions to produce a wax metathesis product having a desired constrained carbon number distribution and/or narrow melting range.

A potential solution for producing a wax metathesis product having a desired constrained carbon number distribution and/or narrow melting range is to consider the carbon number distribution constraints and/or melting range constraints of the desired wax metathesis product and to control the carbon number distribution of the wax metathesis product by selecting a combination of metathesis reaction parameters to produce the wax metathesis product having the desired constrained carbon number distribution and/or narrow melting range. Metathesis reaction parameters which may be selected to produce a wax metathesis product having a desired constrained carbon number distribution and/or narrow melting range may include the olefin feedstock, the metathesis catalyst, and the metathesis reaction conditions.

In an aspect, the present invention is related to a method comprising: 1) controlling a carbon number distribution of a wax metathesis product by selecting metathesis reaction parameters including: a) an olefin feedstock, b) a metathesis catalyst, and c) metathesis reaction conditions; 2) contacting a feedstock composition comprising the olefin feedstock and a catalyst composition comprising the metathesis catalyst; and 3) reacting the olefin feedstock at the metathesis reaction conditions to produce a wax metathesis product having a constrained carbon number distribution and/or having a narrow melting range. In another aspect the method comprises: 1) contacting a feedstock composition comprising a specific olefin feedstock and a catalyst composition comprising a specific metathesis catalyst, and 2) reacting the olefin feedstock at specific metathesis reaction conditions to produce a wax metathesis product having a desired constrained carbon number distribution and/or having a narrow melting range. Features describing the wax metathesis product having a constrained carbon number distribution and/or narrow melting range, the olefin feedstock, the metathesis catalyst, and the metathesis reaction conditions which may be utilized to describe the invention are independently described herein.

In an embodiment, a wax metathesis product having a constrained carbon number distribution with a desired carbon number span may be produced by contacting a feedstock composition comprising an olefin feedstock having a specified carbon number span and a catalyst composition comprising any metal carbene metathesis catalyst described herein, and reacting the olefin feedstock at metathesis reaction conditions comprising any reaction temperature less than 80° C. described herein. In some embodiments wherein the metathesis catalyst may be any metal carbene metathesis catalyst described herein and the metathesis reaction conditions described herein may comprise any reaction temperature less than 80° C., a wax metathesis product having a carbon number span ranging from 2 to 4 may be produced from an olefin feedstock having a carbon number span of 2. In other embodiments wherein the metathesis catalyst may be any metal carbene catalyst described herein and the metathesis reaction conditions described herein may comprise any reaction temperature less than 80° C., a wax metathesis product having a carbon number span ranging from 3 to 6 may be produced from an olefin feedstock having a carbon number span of 3; alternatively, a wax metathesis product having a carbon number span ranging from 5 to 8 may be produced from an olefin feedstock having a carbon number span of 4; alternatively, a wax metathesis product having a carbon number span ranging from 7 to 10 may be produced from an olefin feedstock having a carbon number span of 5; or alternatively, a wax metathesis product having a carbon number span ranging from 8 to 12 may be produced from an olefin feedstock having a carbon number span of 6. In further embodiments wherein the metathesis catalyst may be any metal carbene catalyst described herein and the metathesis reaction conditions described herein may comprise any reaction temperature less than 80° C., a wax metathesis product having a carbon number span ranging from 2 to 6 may be produced from an olefin feedstock having a carbon number span of 2 or 3; alternatively, a wax metathesis product having a carbon number span ranging from 3 to 8 may be produced from an olefin feedstock having a carbon number span of 3 or 4; alternatively, a wax metathesis product having a carbon number span ranging from 5 to 10 may be produced from an olefin feedstock having a carbon number span of 4 or 5; or alternatively, a wax metathesis product having a carbon number span ranging from 7 to 12 may be produced from an olefin feedstock having a carbon number span of 5 or 6.

In an embodiment, a wax metathesis product having a carbon number distribution wherein greater than 85 weight percent, 90 weight percent, or 95 weight percent, of the wax metathesis product has a carbon number ranging from 2FC−2 to 2MC−2, may be produced by contacting a feedstock composition comprising any olefin feedstock described herein and a catalyst composition comprising any metal carbene metathesis catalyst described herein, and reacting the olefin feedstock at metathesis reaction conditions described herein comprising any reaction temperature less than 80° C. In some embodiments, a wax metathesis product having a carbon number distribution wherein less than 15 weight percent, 10 weight percent, or 5 weight percent, of the wax metathesis product has a carbon number less than 2FC−2, may be produced by contacting a feedstock composition comprising any olefin feedstock described herein and a catalyst composition comprising any metal carbene metathesis catalyst described herein, and reacting the olefin feedstock at metathesis reaction conditions described herein comprising any reaction temperature less than 80° C.

In an embodiment, a wax metathesis product having a constrained carbon number distribution with a desired carbon number span may be produced by contacting a feedstock composition comprising an olefin feedstock having a specified carbon number span and a catalyst composition comprising any metal carbene metathesis catalyst described herein, and reacting the olefin feedstock at metathesis reaction conditions described herein comprising any reaction temperature ranging from the melting point of olefin feedstock to 150° C. In an embodiment, wherein the metathesis catalyst may be any metal carbene catalyst described herein and the metathesis reaction conditions described herein may comprise any reaction temperature ranging from the melting point of olefin feedstock to 150° C., a wax metathesis product having a carbon number span ranging from 2 to 3 may be produced from an olefin feedstock having a carbon number span of 1. In other embodiments wherein the metathesis catalyst may be any metal carbene catalyst described herein and the metathesis reaction conditions described herein may comprise any reaction temperature ranging from the melting point of olefin feedstock to 150° C., a wax metathesis product having a carbon number span ranging from 3 to 6 may be produced from an olefin feedstock having a carbon number span of 2; alternatively, a wax metathesis product having a carbon number span ranging from 5 to 10 may be produced from an olefin feedstock having a carbon number span of 3; alternatively, a wax metathesis product having a carbon number span ranging from 7 to 12 may be produced from an olefin feedstock having a carbon number span of 4; alternatively, a wax metathesis product having a carbon number span ranging from 9 to 14 may be produced from an olefin feedstock having a carbon number span of 5; or alternatively, a wax metathesis product having a carbon number span ranging from 11 to 16 may be produced from an olefin feedstock having a carbon number span of 6. In further embodiments wherein the metathesis catalyst may be any metal carbene catalyst described herein and the metathesis reaction conditions described herein may comprise any reaction temperature ranging from the melting point of olefin feedstock to 150° C., a wax metathesis product having a carbon number span ranging from 3 to 10 may be produced from an olefin feedstock having a carbon number span of 2 or 3; alternatively, a wax metathesis product having a carbon number span ranging from 5 to 12 may be produced from an olefin feedstock having a carbon number span of 3 or 4; alternatively, a wax metathesis product having a carbon number span ranging from 7 to 14 may be produced from an olefin feedstock having a carbon number span of 4 or 5; or alternatively, a wax metathesis product having a carbon number span ranging from 9 to 16 may be produced from an olefin feedstock having a carbon number span of 5 or 6.

In an embodiment, a wax metathesis product having a carbon number distribution wherein greater than 70 and less than 90 weight percent, or greater than 70 and less than 85 weight percent, of the wax metathesis product has a carbon number ranging from 2FC−2 to 2MC−2, may be produced by contacting a feedstock composition comprising any olefin feedstock described herein and a catalyst composition comprising any metal carbene metathesis catalyst described herein, and reacting the olefin feedstock at metathesis reaction conditions described herein comprising any reaction temperature ranging from the melting point of olefin feedstock to 150° C. In some embodiments, a wax metathesis product having a carbon number distribution wherein greater than 10 and less than 30 weight percent, or greater than 15 and less than 30 weight percent weight percent, of the wax metathesis product has a carbon number less than 2FC−2, may be produced by contacting a feedstock composition comprising any olefin feedstock described herein and a catalyst composition comprising any metal carbene metathesis catalyst described herein, and reacting the olefin feedstock at metathesis reaction conditions described herein comprising any reaction temperature ranging from the melting point of olefin feedstock to 150° C.

In an embodiment, a wax metathesis product having a constrained carbon number distribution having a desired carbon number span may be produced by contacting a feedstock composition comprising an olefin feedstock having a specified carbon number span and a catalyst composition comprising any metal oxide or metal halide metathesis catalyst described herein, and reacting the olefin feedstock at metathesis reaction conditions described herein comprising any reaction temperature less than 80° C. In some embodiments wherein the metathesis catalyst may be any metal oxide or metal halide metathesis catalyst described herein and the metathesis reaction conditions described herein may comprise any reaction temperature less than 80° C., a wax metathesis product having a carbon number span ranging from 2 to 3 may be produced from an olefin feedstock having a carbon number span of 1. In other embodiments wherein the metathesis catalyst may be any metal oxide or metal halide metathesis catalyst described herein and the metathesis reaction conditions described herein may comprise any reaction temperature less than 80° C., a wax metathesis product having a carbon number span ranging from 3 to 6 may be produced from an olefin feedstock having a carbon number span of 2; alternatively, a wax metathesis product having a carbon number span ranging from 5 to 10 may be produced from an olefin feedstock having a carbon number span of 3; alternatively, a wax metathesis product having a carbon number span ranging from 7 to 12 may be produced from an olefin feedstock having a carbon number span of 4; alternatively, a wax metathesis product having a carbon number span ranging from 9 to 14 may be produced from an olefin feedstock having a carbon number span of 5; or alternatively, a wax metathesis product having a carbon number span ranging from 11 to 16 may be produced from an olefin feedstock having a carbon number span of 6. In further embodiments wherein the metathesis catalyst may be any metal oxide or metal halide metathesis catalyst described herein and the metathesis reaction conditions described herein may comprise any reaction temperature less than 80° C., a wax metathesis product having a carbon number span ranging from 3 to 10 may be produced from an olefin feedstock having a carbon number span of 2 or 3; alternatively, a wax metathesis product having a carbon number span ranging from 5 to 14 may be produced from an olefin feedstock having a carbon number span of 3, 4, or 5; alternatively, a wax metathesis product having a carbon number span ranging from 5 to 12 may be produced from an olefin feedstock having a carbon number span of 3 or 4; alternatively, a wax metathesis product having a carbon number span ranging from 7 to 14 may be produced from an olefin feedstock having a carbon number span of 4 or 5; or alternatively, a wax metathesis product having a carbon number span ranging from 9 to 16 may be produced from an olefin feedstock having a carbon number span of 5 or 6.

In an embodiment, a wax metathesis product having a carbon number distribution wherein greater than 70 and less than 90 weight percent, or greater than 70 and less than 85 weight percent, of the wax metathesis product has a carbon number ranging from 2FC−2 to 2MC−2, may be produced by contacting a feedstock composition comprising any olefin feedstock described herein and a catalyst composition comprising any metal oxide or metal halide metathesis catalyst described herein, and reacting the olefin feedstock at metathesis reaction conditions described herein comprising any reaction temperature less than 80° C. In some embodiments, a wax metathesis product having a carbon number distribution wherein greater than 10 and less than 30 weight percent, or greater than 15 and less than 30 weight percent weight percent, of the wax metathesis product has a carbon number less than 2FC−2, may be produced by contacting a feedstock composition comprising any olefin feedstock described herein and a catalyst composition comprising any metal oxide or metal halide metathesis catalyst described herein, and reacting the olefin feedstock at metathesis reaction conditions described herein comprising any reaction temperature less than 80° C.

In an embodiment, a wax metathesis product having a constrained carbon number distribution having a desired carbon number span may be produced by contacting a feedstock composition comprising an olefin feedstock having a specified carbon number span and a catalyst composition comprising any metal carbene metathesis catalyst described herein, and reacting the olefin feedstock at metathesis reaction conditions described herein comprising any reaction temperature greater than 70° C. In an embodiment, wherein the metathesis catalyst may be any metal carbene metathesis catalyst described herein and the metathesis reaction conditions described herein may comprise any reaction temperature greater than 70° C., a wax metathesis product having a carbon number span ranging from 3 to 6 may be produced from an olefin feedstock having a carbon number span of 1. In other embodiments wherein the metathesis catalyst may be any metal carbene catalyst described herein and the metathesis reaction conditions described herein may comprise any reaction temperature greater than 70° C., a wax metathesis product having a carbon number span ranging from 5 to 10 may be produced from an olefin feedstock having a carbon number span of 2; alternatively, a wax metathesis product having a carbon number span ranging from 8 to 14 may be produced from an olefin feedstock having a carbon number span of 3; alternatively, a wax metathesis product having a carbon number span ranging from 10 to 16 may be produced from an olefin feedstock having a carbon number span of 4; alternatively, a wax metathesis product having a carbon number span ranging from 12 to 18 may be produced from an olefin feedstock having a carbon number span of 5; or alternatively, a wax metathesis product having a carbon number span ranging from 14 to 20 may be produced from an olefin feedstock having a carbon number span of 6.

In the embodiments wherein the metathesis reaction conditions described herein may comprise any reaction temperature less than 80° C., the metathesis reaction conditions may further comprise the presence of a solvent or diluent. In the embodiments wherein the metathesis reaction conditions described herein comprise any reaction temperature greater than the melting point of the olefin feedstock, or alternatively, ranging from the melting point of the olefin feedstock and 150° C., the metathesis reaction conditions described herein may further comprise the presence of a solvent or diluent; or alternatively, comprise the substantial absence of a solvent or diluent. In the embodiments wherein the metathesis reaction conditions described herein comprise any reaction temperature greater than 70° C., the metathesis reaction conditions described herein may further comprise the presence of a solvent or diluent; or alternatively, comprise the substantial absence of a solvent or diluent. The solvent or diluent, if any, may be any metathesis catalyst compatible solvent described herein. The presence of the solvent or diluent may assist in maintaining the reaction solution in a processable state. The term “processable state” refers to solution which can be stirred, pumped, and/or is sufficiently fluid to flow through a column. Consequently, a metathesis reaction solution in a “processable state” does not necessarily refer to a metathesis reaction solution wherein all materials are in the liquid and/or gaseous state. For example, the processable metathesis reaction solution may comprise solid (non-liquid or undissolved) wax particles which do not prevent the ability to stir and/or pump the metathesis reaction solution or impede the metathesis reaction solution flow through a column.

In the embodiments using a catalyst composition comprising, or consisting essentially of, any metal carbene metathesis catalyst described herein, the metal carbene metathesis catalyst may be any ruthenium or molybdenum carbene metathesis catalyst described herein; alternatively, any ruthenium carbene metathesis catalyst described herein; or alternatively, any molybdenum carbene metathesis catalyst described herein. In the embodiments using a catalyst composition comprising, or consisting essentially of, any metal oxide or metal halide metathesis catalyst described herein, the metathesis catalyst may be any metal oxide metathesis catalyst described herein; alternatively, any metal halide metathesis catalyst described herein.

In an embodiment wherein the feedstock composition comprising, or consisting essentially of, an olefin feedstock described herein, the olefin feedstock may comprise, or consists essentially of, any alpha olefin described herein; alternatively, any linear alpha olefin described herein; alternatively, any hydrocarbon alpha olefin described herein; alternatively, any hydrocarbon linear alpha olefin described herein; or alternatively, any normal alpha olefin described herein. Other olefin feedstock features (e.g. carbon number, carbon number range, and average molecular weight, among other features described herein) which may be further utilized to define the olefin feedstock are described herein. Other wax metathesis product features (e.g. olefin type, carbon number, carbon number range, and average molecular weight, among other wax metathesis product features described herein) which may be utilized to further describe the wax metathesis product are described herein. Other wax metathesis reaction conditions (e.g. molar ratio of metathesis catalyst moieties to mole of olefin feedstock, metathesis reaction duration, and molar conversion of olefin feedstock to wax metathesis product, among other metathesis reaction parameters described herein) which may be utilized to further describe the reaction conditions are described herein. Additional method steps (e.g. separating the metathesis catalyst from the wax metathesis product, separating the unconverted olefin feedstock from the wax metathesis product, separating the non-wax metathesis product olefin from the wax metathesis product, separating the wax metathesis product from any solvent or diluent, and hydrogenating the wax metathesis product, among other method steps described herein) which may utilized to further describe the method(s) are described herein.

In an aspect, the invention relates to a wax composition comprising, or consisting essentially of, a wax metathesis product (or wax molecules) produced by any method/process described herein. In some embodiments, the wax metathesis product (or wax molecules) may have any feature of the wax metathesis product described herein. In an embodiment, the wax metathesis product can comprise, or consist essentially of, hydrocarbon molecules.

In an aspect, the invention relates to a hydrocarbon wax composition comprising, or alternatively, consisting essentially of, hydrocarbon wax molecules. The hydrocarbon wax molecules within the hydrocarbon wax composition can be described using either singly or in any combination, the type of molecules (e.g. aliphatic or aromatic, linear or branched, olefinic or saturated, cyclic or acyclic, mono-olefinic or polyolefinic, among others described herein), the number of carbon atoms present in the hydrocarbon wax molecules, the average molecular weight of the hydrocarbon wax molecules, features of a constrained carbon number distribution of the hydrocarbon wax molecules, and/or melting features of the hydrocarbon wax molecules. These features are independently described herein and may be used in any combination to describe the hydrocarbon wax molecules. The constrained carbon number distribution of the hydrocarbon wax molecules can be described using, either singly or in any combination, the weight percentage of hydrocarbon wax molecules having a molecular weight within a specified number of grams/mole of the average molecular weight of the hydrocarbon wax molecules, the weight percentage of hydrocarbon wax molecules having a molecular within a specified molecular weight range, and/or the carbon number span of the hydrocarbon wax molecules, among other features described herein. In some non-limiting embodiments, the hydrocarbon wax molecules can be described using any average molecular weight of the hydrocarbon wax molecules described herein in combination with any feature describing the constrained carbon number of the hydrocarbon wax molecules described herein. The melting features which can be utilized to describe the hydrocarbon wax molecules can include the DSC melting range of the hydrocarbon wax molecules or a combination of the DSC melting point and the DSC melting range of the hydrocarbon wax molecules. Additionally, in a non-limiting embodiment, the hydrocarbon wax molecules can be described using any combination of the constrained carbon number feature(s) of the hydrocarbon wax molecules described herein and the melting feature(s) of the hydrocarbon wax molecules described herein.

In an embodiment, the hydrocarbon wax molecules can comprise, or consist essentially of aliphatic hydrocarbon wax molecules, aromatic hydrocarbon wax molecules, or combinations thereof; alternatively aliphatic hydrocarbon wax molecules; or alternatively, aromatic hydrocarbon wax molecules. In an embodiment, the aliphatic hydrocarbon molecules can comprise, or consist essentially of saturated hydrocarbon wax molecules, olefinic hydrocarbon wax molecules, or combinations thereof; alternatively, saturated hydrocarbon wax molecules; or alternatively, olefinic hydrocarbon wax molecules. In an embodiment, the hydrocarbon wax molecules (whether aliphatic or aromatic, saturated or olefinic, or combinations thereof) can comprise, or consist essentially of linear hydrocarbon wax molecules, branched hydrocarbon wax molecules, or combinations thereof; alternatively, linear hydrocarbon wax molecules; or alternatively, branched hydrocarbon wax molecules. In an embodiment, the hydrocarbon wax molecules (whether aliphatic or aromatic, olefinic or saturated, linear or branched, or combinations thereof) can comprise, or consist essentially of cyclic hydrocarbon wax molecules, acyclic hydrocarbon wax molecules, or combinations thereof; alternatively, cyclic hydrocarbon wax molecules; or alternatively, acyclic hydrocarbon wax molecules In an embodiment, the hydrocarbon wax molecules (whether aliphatic or aromatic, linear or branched, acyclic or cyclic, or combinations thereof) may comprise, or consist essentially of, olefinic hydrocarbon wax molecules. In some other embodiments, the hydrocarbon wax molecules (whether aliphatic or aromatic, linear or branched, cyclic or acyclic, hydrocarbon, mono-olefinic, or combinations thereof) can comprise, or consist essentially of, internal olefinic hydrocarbon wax molecules. In some other embodiments, the hydrocarbon wax molecules (whether aliphatic or aromatic, linear or branched, cyclic or acyclic, hydrocarbon, mono-olefinic, or combinations thereof) can comprise, or consist essentially of, linear internal olefinic hydrocarbon wax molecules. In some other embodiments, the hydrocarbon wax molecules (whether aliphatic or aromatic, linear or branched, cyclic or acyclic, hydrocarbon, mono-olefinic, or combinations thereof) can comprise, or consist essentially of, internal disubstituted olefinic hydrocarbon wax molecules. In other embodiments, the hydrocarbon wax molecules (whether aliphatic or aromatic, linear or branched, cyclic or acyclic, internal, linear internal, internal disubstituted, or combinations thereof) may comprise, or consist essentially of, mono-olefinic hydrocarbon wax molecules. In other embodiments, the hydrocarbon wax molecules (whether linear or branched, cyclic or acyclic, or combinations thereof) may comprise, or consist essentially of, saturated hydrocarbon wax molecules. In yet other embodiments, the hydrocarbon wax molecules (whether aliphatic or aromatic, olefinic or saturated, mono-olefinic, internal, or combinations thereof) comprise, or consist essentially of, linear hydrocarbon wax molecules. In some embodiments, the olefinic hydrocarbon wax molecules (whether aliphatic or aromatic, linear or branched, cyclic or acyclic, mono-olefinic, internal, linear internal, internal disubstituted, or combinations thereof) can comprise, or consist essentially of, olefinic hydrocarbon wax molecules wherein the carbon-carbon double bond is at least 6, 8, 10, or alternatively 12 carbon atoms from each of the two terminal carbon atoms of the longest continuous chain of carbon atoms. In other embodiments, the hydrocarbon wax molecules (whether aliphatic or aromatic, linear or branched, cyclic or acyclic, mono-olefinic, internal, linear internal, internal disubstituted, or combinations thereof) can comprise, or consist essentially of, olefin hydrocarbon wax molecules wherein the two terminal carbon atoms of the longest continuous chain of carbon atoms and the carbon-carbon double bond are separated by at least 6 carbon atoms; alternatively, 8 carbon atoms; alternatively, 10 carbon atoms; or alternatively, 12 carbon atoms. One of ordinary skill in the art will recognize techniques which can be utilized for determining the position of the double bond within the longest contiguous chain of carbon atoms in the olefinic hydrocarbon wax molecules (for example but not limited to ozonolysis or permanganate oxidation of the carbon-carbon double bond followed by analysis of the oxidation products, among other methods). In further embodiments, the hydrocarbon wax molecules (whether aliphatic or aromatic, olefinic or saturated, linear or branched, cyclic or acyclic, mono-olefinic, internal, linear internal, internal disubstituted, or combinations thereof) can comprise an even and odd number of carbon atoms. In yet further embodiments, the hydrocarbon wax molecules (whether aliphatic or aromatic, olefinic or saturated, linear or branched, cyclic or acyclic, mono-olefinic, internal, linear internal, internal disubstituted, or combinations thereof) can comprise, or consist essentially of, an odd number of carbon atoms; or alternatively, an even number of carbon atoms. In yet other embodiments, the olefinic hydrocarbon molecules (whether aliphatic or aromatic, olefinic or saturated, linear or branched, cyclic or acyclic, internal, linear internal, internal disubstituted, or combinations thereof) can comprise, or consist essentially of, mono-olefinic hydrocarbon molecules.

In an embodiment, the hydrocarbon wax molecules have greater than 30 carbon atoms; alternatively, greater than 34 carbon atoms; alternatively, greater than 40 carbon atoms; alternatively, greater than 45 carbon atoms; or alternatively, greater than 50 carbon atoms. In some embodiments, the hydrocarbon wax molecules have from 30 to 100 carbon atoms; alternatively, from 30 to 80 carbon atoms; alternatively, from 30 to 60 carbon atoms; alternatively, from 30 to 50 carbon atoms; alternatively, from 30 to 40 carbon atoms; alternatively, from 34 to 80 carbon atoms; alternatively, from 34 to 60 carbon atoms; alternatively, from 34 to 50 carbon atoms; alternatively, from 40 to 80 carbon atom; alternatively, from 40 to 70 carbon atoms; alternatively, from 40 to 60 carbon atoms; alternatively, from 45 to 60 carbon atoms; alternatively from 50 to 80 carbon atoms; or alternatively from 50 to 70 carbon atoms.

In an embodiment, the hydrocarbon wax molecules have an average molecular weight greater than 400 grams/mole; alternatively, greater than 450 grams/mole; alternatively, greater than 500 grams/mole; alternatively, greater than 600 grams/mole; or alternatively, greater than 800 grams/mole. In some embodiments, the hydrocarbon wax molecules have an average molecular weight ranging from 400 to 1500 grams/mole; alternatively, ranging from 450 to 1200 grams/mole; alternatively, ranging from 400 to 1000 grams/mole; alternatively, ranging from 500 to 1000 grams/mole; alternatively, ranging from 400 to 850 grams/mole; alternatively, ranging from 400 to 560 grams/mole; alternatively, ranging from 430 to 530 grams/mole; alternatively, ranging from 450 to 500 grams/mole; alternatively, ranging from 500 to 850 grams/mole; alternatively, ranging from 500 to 660 grams/mole; alternatively, ranging from 530 to 620 grams/mole; alternatively, ranging from 550 to 600 grams/mole; alternatively, ranging from 600 to 820 grams/mole; alternatively, ranging from 640 to 780 grams/mole; alternatively, ranging from 680 to 740 grams/mole; alternatively, ranging from 750 to 1200 grams/mole; alternatively, ranging from 800 to 1150 grams/mole; alternatively, ranging from 850 to 1100 grams/mole; or alternatively, ranging from 900 to 1050 grams/mole.

In an embodiment, the hydrocarbon wax molecules have a constrained carbon number distribution as described herein and/or a narrow melting criteria described herein.

In an embodiment, the hydrocarbon wax molecules having a constrained carbon number distribution have a distribution of hydrocarbon wax molecules wherein greater than 70 weight percent of the hydrocarbon wax molecules have a molecular weight within 42 grams/mole of the average molecular weight of the hydrocarbon wax molecules; alternatively, within 35 grams/mole of the average molecular weight of the hydrocarbon wax molecules; alternatively, within 28 grams/mole of the average molecular weight of the hydrocarbon wax molecules; or alternatively, within 21 grams/mole of the average molecular weight of the hydrocarbon wax molecules. In an embodiment, the hydrocarbon wax molecules having a constrained carbon number distribution have a distribution of hydrocarbon wax molecules wherein greater than 80 weight percent of the hydrocarbon wax molecules have a molecular weight within 49 grams/mole of the average molecular weight of the hydrocarbon wax molecules; alternatively, within 42 grams/mole of the average molecular weight of the hydrocarbon wax molecules; alternatively, within 35 grams/mole of the average molecular weight of the hydrocarbon wax molecules; or alternatively, within 28 grams/mole of the average molecular weight of the hydrocarbon wax molecules. In an embodiment, the hydrocarbon wax molecules having a constrained carbon number distribution have a molecular weight distribution wherein greater than 85 weight percent of the hydrocarbon wax molecules have a molecular weight within 56 grams/mole of the average molecular weight of the hydrocarbon wax molecules; alternatively, within 49 grams/mole of the average molecular weight of the hydrocarbon wax molecules; alternatively, within 42 grams/mole of the average molecular weight of the hydrocarbon wax molecules; or alternatively, within 35 grams/mole of the average molecular weight of the hydrocarbon wax molecules. In an embodiment, the hydrocarbon wax molecules having a constrained carbon number distribution have a molecular weight distribution of hydrocarbon wax molecules wherein greater than 90 weight percent of the hydrocarbon wax molecules have a molecular weight within 63 grams/mole of average molecular weight of the hydrocarbon wax molecules; alternatively, within 56 grams/mole of the average molecular weight of the hydrocarbon wax molecules; alternatively, within 49 grams/mole of the average molecular weight of the hydrocarbon wax molecules; or alternatively, within 42 grams/mole of the average molecular weight of the hydrocarbon wax molecules. In an embodiment, the hydrocarbon wax molecules having a constrained carbon number distribution have a molecular weight distribution of hydrocarbon wax molecules wherein greater than 95 weight percent of the hydrocarbon wax molecules have a molecular weight within 63 grams/mole of the average molecular weight of the hydrocarbon wax molecules; alternatively, within 56 grams/mole of the average molecular weight of the hydrocarbon wax molecules; or alternatively, within 49 grams/mole of the average molecular weight of the hydrocarbon wax molecules; or alternatively, within 42 grams/mole of the average molecular weight of the hydrocarbon wax molecules.

In an embodiment, the hydrocarbon wax molecules have a molecular weight distribution wherein a specified percentage of hydrocarbon wax molecules have a molecular weight within a specified molecular weight range. In some embodiments, greater than 90 weight percent of the hydrocarbon wax molecules have a molecular weight ranging from 500 to 660 grams/mole; alternatively, greater than 90 weight percent of the hydrocarbon wax molecules have a molecular weight ranging from 630 to 800 grams/mole; or alternatively, greater than 90 weight percent of the hydrocarbon molecules have a molecular weight ranging from 750 to 1200 grams/mole.

In an embodiment, the hydrocarbon wax molecules having a constrained carbon number distribution have a carbon number span less than 15; alternatively, less than 13; alternatively, less than 11: alternatively, less than 9; or alternatively, less than 7. In some embodiments, the hydrocarbon wax molecules having a constrained carbon number distribution have a carbon number span greater than 1 and less than 15; alternatively greater than 1 and less than 13; alternatively, greater than 1 and less than 11; alternatively, greater than 1 and less than 9; or alternatively, greater than 1 and less than 7. In other embodiments, the hydrocarbon wax molecules having a constrained carbon number distribution have a carbon number span greater than 2 and less than 15; alternatively greater than 2 and less than 13; alternatively, greater than 2 and less than 11; alternatively, greater than 2 and less than 9; or alternatively, greater than 2 and less than 7. In other embodiments, the hydrocarbon wax molecules having a constrained carbon number distribution have a carbon number span greater than 3 and less than 15; alternatively greater than 3 and less than 13; alternatively, greater than 3 and less than 11; alternatively, greater than 3 and less than 9; or alternatively, greater than 3 and less than 7. In other embodiments, the hydrocarbon wax molecules having a constrained carbon number distribution have a carbon number span greater than 4 and less than 15; alternatively greater than 4 and less than 13; alternatively, greater than 4 and less than 11; of alternatively, greater than 4 and less than 9. In further embodiments, the hydrocarbon wax molecules having a constrained carbon number distribution have a carbon number span greater than 5 and less than 15; alternatively greater than 5 and less than 13; alternatively, greater than 5 and less than 11; or alternatively, greater than 5 and less than 9.

In an embodiment, the hydrocarbon wax molecules having a narrow DSC melting range and/or constrained carbon number distribution may have a DSC melting range less than 20° C.; alternatively, less than 15° C.; alternatively, less than 13° C.; alternatively, less than 12° C.; alternatively, less than 11° C.; or alternatively, less than 10° C. In some embodiments, the hydrocarbon wax molecules having a narrow DSC melting range and/or constrained carbon number distribution may have a DSC melting range greater than 3° C. and less than 20° C.; alternatively, greater than 3° C. and less than 15° C.; alternatively, greater than 3° C. and less than 13° C.; alternatively, greater than 3° C. and less than 12° C.; alternatively, greater than 3° C. and less than 11; alternatively, greater than 3° C. and less than 10° C.; alternatively, greater than 5° C. and less than 20° C.; alternatively, greater than 5° C. and less than 15° C.; alternatively, greater than 5° C. and less than 13° C.; alternatively, greater than 5° C. and less than 12° C.; alternatively, greater than 5° C. and less than 11; or alternatively, greater than 5° C. and less than 10° C.

In an embodiment, the hydrocarbon wax molecules having any DSC melting range described herein can have a DSC melting point greater than 50° C.; alternatively greater than 60° C.; or alternatively, greater than 65° C. In some embodiments, the hydrocarbon wax molecules having any DSC melting point described herein can have a DSC melting point ranging from 50° C. to 80° C.; alternatively, ranging from 55° C. to 75° C.; alternatively, ranging from 60° C. to 70° C.; alternatively, ranging from 60° C. to 90° C.; alternatively, ranging from 65° C. to 85° C.; alternatively, ranging from 70° C. to 80° C.; alternatively, ranging from 70° C. to 100° C.; alternatively, ranging from 75° C. to 95° C.; alternatively, ranging from 80° C. to 90° C.; alternatively, ranging from 80° C. to 110° C.; alternatively, ranging from 85° C. to 105° C.; alternatively, ranging from 90° C. to 100° C.; alternatively, ranging from 90° C. to 120° C.; alternatively, ranging from 95° C. to 115° C.; alternatively, ranging from 100° C. to 110° C.; or alternatively, ranging from 100° C. to 125° C.

Generally, the hydrocarbon wax molecules of the hydrocarbon wax composition can have any combination of DSC melting point as described herein and DSC melting range as described herein. In some exemplary non-limiting embodiments, the hydrocarbon wax molecules of the hydrocarbon wax composition can have a DSC melting point ranging from 65° C. to 85° C. and a DSC melting range less than 15° C.; alternatively, have a DSC melting point ranging from 75° C. to 95° C. and a DSC melting range less than 15° C.; have a DSC melting point ranging from 85° C. to 105° C. and a DSC melting range less than 15° C. Other potential combinations of the DSC melting point range and DSC melting point are evident from the DSC melting ranges described herein and the DSC melting points described herein.

The hydrocarbon wax molecules of the hydrocarbon wax composition can be described using any combination of the type of hydrocarbon wax molecules, the number of carbon atoms present in the hydrocarbon wax molecules, the features of the constrained carbon number distribution, the average molecular weight of the hydrocarbon wax molecules, and/or melting features of the hydrocarbon wax molecules. Several exemplary non-limiting combinations for describing the hydrocarbon wax molecules include the number of carbon atoms present in the hydrocarbon wax molecules, the carbon number span of the hydrocarbon wax molecules, and the DSC melting range of the hydrocarbon wax molecules; alternatively, the number of carbon atoms present in the hydrocarbon wax molecules, the carbon number span of the hydrocarbon wax molecules, the DSC melting range of the hydrocarbon wax molecules, and the DSC melting point of the hydrocarbon wax molecules; alternatively, the number of carbon atoms present in the hydrocarbon wax molecules, the carbon number span of the hydrocarbon wax molecules and the percentage of hydrocarbon wax molecules having a molecular weight within a specified number of grams/mole of the average molecular weight of the hydrocarbon wax molecules; alternatively, the number of carbon atoms present in the hydrocarbon wax molecules, the carbon number span of the hydrocarbon wax molecules, the percentage of hydrocarbon wax molecules having a molecular weight within a specified number of grams/mole of the average molecular weight of the hydrocarbon wax molecules, and the DSC melting range of the hydrocarbon wax molecules; alternatively, the number of carbon atoms present in the hydrocarbon wax molecules, the carbon number span of the hydrocarbon wax molecules, the percentage of hydrocarbon wax molecules having a molecular weight within a specified number of grams/mole of the average molecular weight of the hydrocarbon wax molecules, the DSC melting point of the hydrocarbon wax molecules, and the DSC melting range of the hydrocarbon wax molecules; alternatively, the average molecular weight of the hydrocarbon wax molecules, the carbon number span of the hydrocarbon wax molecules, the percentage of hydrocarbon wax molecules having a molecular weight within a specified number of grams/mole of the average molecular weight of the hydrocarbon wax molecules, and the DSC melting range of the hydrocarbon wax molecules; alternatively, the average molecular weight of the hydrocarbon wax molecules, the carbon number span of the hydrocarbon wax molecules, and the DSC melting range of the hydrocarbon wax molecules; alternatively, the average molecular weight of the hydrocarbon wax molecules, the carbon number span of the hydrocarbon wax molecules, the DSC melting range of the hydrocarbon wax molecules, and the DSC melting point of the hydrocarbon wax molecules; alternatively, the type of hydrocarbon wax molecules present, the number of carbon atoms present in the hydrocarbon wax molecules, the carbon number span of the hydrocarbon wax molecules and the percentage of hydrocarbon wax molecules having a molecular weight within a specified number of grams/mole of the average molecular weight of the hydrocarbon wax molecules; alternatively, the type of hydrocarbon wax molecules present, the number of carbon atoms present in the hydrocarbon wax molecules, the carbon number span of the hydrocarbon wax molecules, the percentage of hydrocarbon wax molecules having a molecular weight within a specified number of grams/mole of the average molecular weight of the hydrocarbon wax molecules, and the DSC melting point of the hydrocarbon wax molecules; or alternatively, the type of hydrocarbon wax molecules present, the number of carbon atoms present in the hydrocarbon wax molecules, the carbon number span of the hydrocarbon wax molecules, the percentage of hydrocarbon wax molecules having a molecular weight within a specified number of grams/mole of the average molecular weight of the hydrocarbon wax molecules, the DSC melting point of the hydrocarbon wax molecules, and the DSC melting range of the hydrocarbon wax molecules.

In an exemplary non-limiting embodiment, the hydrocarbon wax molecules comprise, or consist essentially of, olefinic hydrocarbon wax molecules having a) greater than 30 carbon atoms, b) a carbon number span greater than 1 and less than 15, and c) a DSC melting range less than 15° C. In another exemplary non-limiting embodiment, the hydrocarbon wax molecules comprise, or consist essentially of, olefinic hydrocarbon wax molecules having a) greater than 30 carbon atoms, b) a carbon number span greater than 1 and less than 15, and c) a distribution of hydrocarbon wax molecules wherein greater than 85 weight percent of the hydrocarbon wax molecules have a molecular weight within 49 grams/mole of the average molecular weight of the hydrocarbon wax molecules.

In further exemplary non-limiting embodiments, the hydrocarbon wax molecules comprise, or consist essentially of, olefinic hydrocarbon wax molecules wherein the hydrocarbon wax molecules having a) greater than 30 carbon atoms, b) an average molecular weight greater than 450 grams/mole, c) a carbon number span greater than 1 and less than 15, and d) a distribution of hydrocarbon wax molecules wherein greater than 85 weight percent of the hydrocarbon wax molecules have a molecular weight within 49 grams/mole of the average molecular weight of the hydrocarbon wax molecules; alternatively, a) greater than 30 carbon atoms, b) an average molecular weight greater than 450 grams/mole, c) a carbon number span greater than 1 and less than 15, d) a distribution of hydrocarbon wax molecules wherein greater than 85 weight percent of the hydrocarbon wax molecules have a molecular weight within 49 grams/mole of the average molecular weight of the hydrocarbon wax molecules, and e) a DSC melting range less than 15° C.; alternatively, a) greater than 30 carbon atoms, b) a carbon number span greater than 1 and less than 15, c) a distribution of hydrocarbon wax molecules wherein greater than 85 weight percent of the hydrocarbon wax molecules have a molecular weight within 49 grams/mole of the average molecular weight of the hydrocarbon wax molecules, d) a DSC melting point greater than 50° C., and e) a DSC melting range less than 15° C.; or alternatively, a) an average molecular weight greater than 450 grams/mole, b) greater than 30 carbon atoms, c) a carbon number span greater than 1 and less than 15, d) a distribution of hydrocarbon wax molecules wherein greater than 85 weight percent of the hydrocarbon wax molecules have a molecular weight within 49 grams/mole of the average molecular weight of the hydrocarbon wax molecules, and e) a DSC melting range less than 15° C.

In some other exemplary non-limiting embodiments, the hydrocarbon wax molecules comprise, or consist essentially of, olefinic hydrocarbon wax molecules having a) greater than 30 carbon atoms, b) a carbon number span greater than 1 and less than 15, c) a distribution of hydrocarbon wax molecules wherein greater than 85 weight percent of the hydrocarbon wax molecules have a molecular weight within 49 grams/mole of the average molecular weight of the hydrocarbon wax molecules and wherein greater than 90 weight percent of the hydrocarbon wax molecules have a molecular weight ranging from 500 to 660 grams/mole, d) a DSC melting point ranging from 65° C. to 85° C., and e) a DSC melting range less than 15° C.; alternatively, the hydrocarbon molecules comprise, or consist essentially of, saturated hydrocarbon wax molecules having a) greater than 30 carbon atoms, b) a carbon number span greater than 1 and less than 15, c) a distribution of hydrocarbon wax molecules wherein greater than 85 weight percent of the hydrocarbon wax molecules have a molecular weight within 49 grams/mole of the average molecular weight of the hydrocarbon wax molecules and wherein greater than 90 weight percent of the hydrocarbon wax molecules have a molecular weight ranging from 500 to 660 grams/mole, d) a DSC melting point ranging from 75° C. to 95° C., and e) a DSC melting range less than 15° C.; alternatively, the hydrocarbon molecules comprise, or consist essentially of, olefinic hydrocarbon wax molecules having a) greater than 30 carbon atoms, b) a carbon number span greater than 1 and less than 15, c) a distribution of hydrocarbon wax molecules wherein greater than 85 weight percent of the hydrocarbon wax molecules have a molecular weight within 49 grams/mole of the average molecular weight of the hydrocarbon wax molecules and wherein greater than 90 weight percent of the hydrocarbon wax molecules have a molecular weight ranging from 630 to 800 grams/mole, d) a DSC melting point ranging from 75° C. to 95° C., and e) a DSC melting range less than 15° C.; or alternatively, the hydrocarbon wax molecules comprise, or consist essentially of, saturated hydrocarbon wax molecules having a) greater than 30 carbon atoms, b) a carbon number span greater than 1 and less than 15, c) a distribution of hydrocarbon wax molecules wherein greater than 85 weight percent of the hydrocarbon wax molecules have a molecular weight within 49 grams/mole of the average molecular weight of the hydrocarbon wax molecules and wherein greater than 90 weight percent of the hydrocarbon wax molecules have a molecular weight ranging from 630 to 800 grams/mole, d) a DSC melting point ranging from 85° C. to 105° C., and e) a DSC melting range less than 15° C. Other combinations for describing the hydrocarbon wax molecules of the hydrocarbon wax composition using the types of hydrocarbon wax molecules present, the number of carbon atoms in the hydrocarbon wax molecules, the carbon number span of the hydrocarbon wax molecules, the percentage of hydrocarbon wax molecules having an molecular weight within a specified number of grams/mole of the average molecular weight of the hydrocarbon wax molecules, the average molecular weight of the hydrocarbon wax molecules, DSC melting range and/or DSC melting range are easily envisioned and contemplated by this description.

In some embodiments, the hydrocarbon wax molecules of the hydrocarbon composition may be produced utilizing the method(s) described herein. Consequently, the hydrocarbon wax molecules of the hydrocarbon wax composition may be described as a product of any processes (i.e. method) for producing a wax metathesis product described herein wherein the olefin feedstock comprises, or consists essentially of, any hydrocarbon olefin feedstock described herein.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Use of the term “optionally” with respect to any element is intended to mean that the subject element is required, or alternatively, is not required. Both alternatives are intended to be within the scope. Use of broader terms such as “comprises,” “includes,” 37 having,” etc. should be understood to provide support for narrower terms such as “consisting essentially of,” “consisting of,” “comprised substantially of,” etc. . . .

The scope of protection is not limited by the description set out within but is only limited by the claims which follow, that scope including all equivalents of the subject matter of the claims. Each and every claim is incorporated into the specification as an embodiment of the present invention. Consequently, the claims are a further description and are an addition to the preferred embodiments of the present invention. The discussion of a reference within this application is not an admission that it is prior art to the present invention(s), especially any reference that may have a publication date after the priority date of this application. The disclosures of all patents, patent applications, and publications cited herein are hereby incorporated by reference, to the extent that they provide exemplary, procedural or other details supplementary to those set forth herein.

The following examples are included to demonstrate specific embodiments of the invention. Those of skill in the art should appreciate that the techniques disclosed in the examples represent techniques discovered to function well in the practice of the invention. However, in light of the present disclosure, those of skill in the art should will appreciate the changes that can be made in the specific disclosed embodiments and still obtain similar results that do not depart from the spirit and scope of the invention.

### EXAMPLES

In the examples, normal alpha olefins were subjected to metathesis to produce a wax metathesis product. The wax metathesis products were subsequently hydrogenated. Typical properties of the normal alpha olefin feedstocks prior to metathesis are provided in Table 1.

TABLE 1
Typical Properties of Some Commercial Normal Alpha Olefins

| | Typical Value (Typical Range) | | |
|---|---|---|---|
| | Alpha | Alpha | Alpha |
| Characteristic | Olefin C₁₈ | Olefin C₂₀₋₂₄ | Olefin C₂₆₋₂₈ |

Olefin Type Distribution (¹H-NMR)

| | | | | | | |
|---|---|---|---|---|---|---|
| % Normal Alpha | 91.5 | (89-93) | 89 | (83-92) | 86 | (70-82) |
| Olefins | | | | | | |
| % Vinylidenes | 6.1 | (5-7) | 8 | (6-15) | 13 | (11-17) |
| % Internal Olefins | 0.2 | (0.2-0.5) | 0.3 | (0.2-0.5) | 0.3 | (0.2-0.8) |
| % Paraffin | 0.3 | (0.2-0.5) | 0.3 | (0.2-0.5) | 0.2 | (0.2-0.5) |

Carbon Number Distribution (Gas Chromatography)

| | | | |
|---|---|---|---|
| C₁₆ | <1.0 | | |
| C₁₈ | 98.6 | <0.5 | |
| C₂₀ | <1.0 | 40.6 | |
| C₂₂ | | 35.3 | |
| C₂₄ | | 22.4 | <1.2 |
| C₂₆ | | 1.0 | 56.9 |
| C₂₈ | | <0.10 | 39.4 |
| C₃₀ | | | 2.6 |
| Carbon Number | 1 | 5 | 3 |
| Span | | | |

Properties of the wax metathesis products and the hydrogenated wax metathesis products are provided in Table 2 and Table 3.

### Metathesis Reaction

The normal alpha olefin, 300 grams, was placed in a 1000 mL three-neck flask equipped with a heating mantel, thermocouple, and a magnetic stirrer. The alpha olefin was heated to 80° C. under a nitrogen atmosphere. When the alpha olefin attained 80° C., the desired metathesis catalyst, 100 mg, 1,3-bis-(2,4,6-trimethylphenyl)-2-(imidazolidinyl-idene)(phenylmethylene)dichloro(tricyclohexylphosphine)ruthenium, was charged into the 1000 mL three-necked flask. The addition of the metathesis catalyst resulted in the instant reaction with evolution of ethylene gas. The metathesis reaction was allowed to continue for three hours. The contents of three-necked flask were allowed to cool and the catalyst separated from the metathesis product by filtration. The wax metathesis product was purified by recrystallization from dodecene and a subsequent recrystallization from heptane. The product was air dried at room temperature for two days. In some instances, the final product was further purified by a filtration through a bed of aluminum oxide powder (alumina) to remove metathesis catalyst residuals.

### Hydrogenation of the Wax Metathesis Product

The wax metathesis product, 100 g, and Crossfield HTC-500 hydrogenation catalyst (nickel and nickel oxide on alumina pellets), 5 g, were charged to an autoclave reactor. The autoclave reactor was closed and the atmosphere in the reactor was purged with nitrogen and vented to ambient pressure. The autoclave reactor was then heated to 150° C. with gentle stirring. When the autoclave reactor attained 150° C., hydrogen was introduced to the autoclave reactor to a pressure of ranging from 2.4 mPa gauge (350 psig) to 3.4 mPa gauge (500 psig), and the temperature increased to 195° C. The autoclave reactor temperature and the hydrogen pressure were maintained for the duration of the reaction, typically 14-16 hours. The reactor was then allowed to cool to 100-110° C. and slowly vented. The hydrogenated wax metathesis product was decanted from the catalyst. Residual hydrogenation catalyst was removed by vacuum filtration.

### Wax Metathesis Product and Hydrogenated Wax Metathesis Product Analysis

The wax metathesis product and hydrogenated wax metathesis products were analyzed using Gas Chromatography (GC). The GC analysis was conducted on Hewlett Packard HP6890 System, using a Varian WCOT Ulti-metal 10 m×0.53 mm column coated with HT SIMDIST CB, DF=0.17 μm. The analysis was performed with an on-column splitless injection with a 10 ml/min helium carrier gas flow rate. The detector for the analysis was a flame ionization detector operated at 420° C. with a H₂ flow of 40.0 mL/min, an air flow of 450 mL/min, and a helium makeup flow of 20 mL/min. The injection port temperature was allowed to track the oven temperature. The analysis oven temperature was programmed for an initial temperature of 100° C. for 0 minutes, a first temperature ramp of 100° C. to 180° C. at a rate of 15° C./min immediately followed by a second temperature ramp of 180° C. to 420° C. at a rate of 10° C./min. The sample was prepared for analysis by dissolving 100 mg of the wax metathesis product, or hydrogenated wax metathesis product, in 25 mL of xylene. The sample size injected onto the GC analysis column was 0.15 microliter.

The carbon numbers present in the wax metathesis product, or hydrogenated wax metathesis product, was determined by comparison to an ASTM 5442 C₁₂-C₆₀ Paraffin Standard supplied by Aldrich. The average molecular weight and other weight or molecular weight related properties of the wax metathesis product and hydrogenated wax metathesis product was then calculated from the weight compositions of the integrated peaks of the GC analysis using routine techniques known to those having ordinary skill in the art.

DSC analyses were conducted on Perkin-Elmer DSC-7, using ASTM D 3418, with two heating scans with an interceding cooling scan. The first heating scan and second heating scans were conducted with a heating rate of 20° C./min from −20° C. to 120° C. The interceding cooling scan was conducted at rate of 20° C./min from 120° C. to −20° C. The “DSC melting point” was taken to be the temperature within the DSC melting transition of the second heating scan having the greatest heat flow. The “DSC melting range” was taken to be the temperature difference between the onset and end point of the DSC melting transition of the second heating scan as determined by the crossing point between the baseline and normal slope of the DSC melting transition peak.

TABLE 2
Properties of the Wax Metathesis Product and Hydrogenated Wax Metathesis Product
Produced from the Metathesis of Normal Alpha Olefins.

| | | | | | Number of grams/mole from the average | | |
|---|---|---|---|---|---|---|---|
| | | | | | molecular weight which encompass | | |
| | | Melting Range | Average | | the indicated weight percent | | |
| | | (Melting Point End | Molecular | Carbon | of the wax metathesis product | | |
| | Melting | Point-Melting Point | Weight | Number | 90 weight | 80 weight | 70 weight |
| | Point | Onset —) | (grams/mole) | Span | percent | percent | percent |
| Alpha Olefin C₁₈ | 64° C. | 11° C. | 475 | 2 | — | — | — |
| Wax Metathesis Product | | (59° C.-70° C.) | | | | | |
| Alpha Olefin C₁₈ Hydrogenated | 74° C. | 12° C. | 477 | 2 | — | — | — |
| Wax Metathesis Product | | (67° C.-79° C.) | | | | | |
| Alpha Olefin C₂₀₋₂₄ | 69° C. | 10° C. | 580 | 10 | 49 grams/mole | 42 grams/mole | 35 grams/mole |
| Wax Metathesis Product | | (62° C.-72° C.) | | | | | |
| Alpha Olefin C₂₀₋₂₄ | 84° C. | 10° C. | 576 | 10 | 49 grams/mole | 42 grams/mole | 35 grams/mole |
| Hydrogenated Wax | | (78° C.-88° C.) | | | | | |
| Metathesis Product | | | | | | | |
| Alpha Olefin C₂₆₋₂₈ | 82° C. | 10° C. | 712 | 8 | 49 grams/mole | 35 grams/mole | 28 grams/mole |
| Wax Metathesis Product | | (77° C.-87° C.) | | | | | |
| Alpha Olefin C₂₆₋₂₈ | 95° C. | 8° C. | 714 | 8 | 49 grams/mole | 35 grams/mole | 28 grams/mole |
| Hydrogenated Wax | | (92° C.-100° C.) | | | | | |
| Metathesis Product | | | | | | | |

TABLE 3
Carbon Number Distribution of the Wax Metathesis Product and Hydrogenated Wax Metathesis Product.

| | Carbon Number (wt. % by Gas Chromatography) | | | | | | | | | | | | |
|---|---|---|---|---|---|---|---|---|---|---|---|---|---|
| | C₃₂ | C₃₃ | C₃₄ | C₃₅ | C₃₆ | | | | | | | | |
| Alpha Olefin C₁₈ | 0.70 | 6.74 | 91.44 | 0.36 | 0.76 | | | | | | | | |
| Wax Metathesis Product | | | | | | | | | | | | | |
| Alpha Olefin C₁₈ Hydrogenated | 0.56 | 6.17 | 92.19 | 0.30 | 0.78 | | | | | | | | |
| Wax Metathesis Product | | | | | | | | | | | | | |
| | C₃₆ | C₃₇ | C₃₈ | C₃₉ | C₄₀ | C₄₁ | C₄₂ | C₄₃ | C₄₄ | C₄₅ | C₄₆ | | |
| Alpha Olefin C₂₀₋₂₄ | | 0.65 | 6.29 | 5.83 | 24.08 | 10.23 | 27.51 | 6.49 | 13.56 | 2.04 | 3.32 | | |
| Wax Metathesis Product | | | | | | | | | | | | | |
| Alpha Olefin C₂₀₋₂₄ Hydrogenated | 0.27 | 2.29 | 11.29 | 8.08 | 24.87 | 9.90 | 23.7 | 4.96 | 10.73 | 1.49 | 2.46 | | |
| Wax Metathesis Product | | | | | | | | | | | | | |
| | C₄₄ | C₄₅ | C₄₆ | C₄₇ | C₄₈ | C₄₉ | C₅₀ | C₅₁ | C₅₂ | C₅₃ | C₅₄ | C₅₅ | C₅₆ |
| Alpha Olefin C₂₆₋₂₈ | | | 0.42 | 1.86 | 6.09 | 13.62 | 24.01 | 15.54 | 22.76 | 6.16 | 7.61 | 1.01 | 0.91 |
| Wax Metathesis Product | | | | | | | | | | | | | |
| Alpha Olefin C₂₆₋₂₈ Hydrogenated | | 0.19 | 0.69 | 2.23 | 6.34 | 13.56 | 23.65 | 15.27 | 22.48 | 6.10 | 7.56 | 1.01 | 0.93 |
| Wax Metathesis Product | | | | | | | | | | | | | |

All of the compositions and methods disclosed and claimed herein can be made and executed without undue experimentation in light of the present disclosure. While the compositions and methods of this invention have been described in terms of particular embodiments, it will be apparent to those of skill in the art that variations may be applied to the compositions and methods and in the steps or in the sequence of steps of the methods described herein without departing from the concept, spirit and scope of the invention as defined by the appended claims.

What is claimed is:

1. A method comprising: 1) contacting a feedstock composition comprising an olefin feedstock comprising olefins having at least 16 carbon atoms and a catalyst composition comprising a metathesis catalyst, 2) reacting the olefin feedstock at metathesis reaction conditions selected to produce a wax metathesis product having a constrained carbon number distribution wherein the carbon number span is greater than 1 and less than 15.

2. The method of claim 1, wherein the olefin feedstock is an olefinic hydrocarbon consisting essentially of a linear alpha olefin, a branched alpha olefin having a branch at the 3-position or higher with respect to the olefin double bond, or mixtures thereof.

3. The method of claim 1, wherein the olefin feedstock consists essentially of normal alpha olefins.

4. The method of claim 1, wherein the olefin feedstock has a carbon number span greater than 1 and less than 8 carbon atoms.

5. The method of claim 1, wherein the metathesis reaction conditions comprise a metathesis reaction temperature ranging from the melting point of the olefin feedstock to 120° C.

6. The method of claim 1, wherein less than 30 weight percent of the wax metathesis product has carbon number less than 2FC−2.

7. The method of claim 1, wherein the metathesis catalyst is selected from a ruthenium carbene metathesis catalyst and a molybdenum carbene metathesis catalyst.

8. The method of claim 1, wherein the metathesis catalyst is dichloro(phenylmethylene) bis(tricyclohexylphosphine) ruthenium or 1,3-bis-(2,4,6-trimethylphenyl)-2-(imidazolidinylidene)(phenylmethylene)dichloro(tricyclohexylphosphine)ruthenium.

9. The method of claim 1, wherein the olefin feedstock has a carbon number span of 3 or 4, the metathesis catalyst is a metal carbene metathesis catalyst, the metathesis reaction conditions comprise a reaction temperature ranging from the melting point of the olefin feedstock and 120° C., and the wax metathesis product has a carbon number span ranging from 5 to 12.

10. The method of claim 1, wherein the process further comprises separating the wax metathesis catalyst from the wax metathesis product.

11. The method of claim 1, wherein the wax metathesis product is separated from a non-wax metathesis product olefin.

12. The method of claim 1, wherein the wax metathesis product is separated from the olefin feedstock by recrystallization.

13. The method of claim 1, wherein the wax metathesis product is separated from the olefin feedstock by fractionational crystallization.

14. The method of claim 1, wherein the wax metathesis product is separated from the olefin feedstock by distillation.

15. The method of claim 1, wherein the process further comprises hydrogenating the wax metathesis product.

16. A wax composition comprising hydrocarbon wax molecules having a) greater than 30 carbon atoms, b) a carbon number span greater than 1 and less than 15, and c) a distribution of hydrocarbon wax molecules wherein 85 weight % of the hydrocarbon wax molecules have molecular weight within 49 grams/mole of the average molecular weight of the hydrocarbon wax molecules.

17. The wax composition of claim 16, wherein the hydrocarbon wax molecules have a DSC melting point greater than 50° C. and a DSC melting range of less than 15° C.

18. The wax composition of claim 16, wherein the hydrocarbon wax molecules comprise olefinic hydrocarbon wax molecules.

19. The wax composition of claim 16, wherein the hydrocarbon wax molecules comprise saturated hydrocarbon wax molecules.

20. The wax composition of claim 16, wherein the hydrocarbon wax molecules consist essentially of linear wax molecules.

21. The wax composition of claim 16, wherein the hydrocarbon wax molecules are olefinic and wherein carbon-carbon double bond of the olefinic hydrocarbon wax molecules is at least 6 carbon atoms from each terminal carbon atom of the longest contiguous chain of carbon atoms.

22. The wax composition of claim 16, wherein the hydrocarbon wax molecules have even and odd number of carbon atoms.

23. The wax composition of claim 16, wherein 90 weight percent of the hydrocarbon wax molecules have a molecular weight ranging from 500 to 660 grams/mole.

24. The wax composition of claim 23, wherein the hydrocarbon wax molecules comprise olefinic hydrocarbon wax molecules and the hydrocarbon wax molecules have a DSC melting point ranging from 65° C. to 85° C. and a DSC melting range of less than 15° C.

25. The wax composition of claim 23, wherein the hydrocarbon wax molecules consist essentially of saturated hydrocarbon wax molecules and the hydrocarbon wax molecules have a DSC melting point ranging from 75 to 95° C. and a DSC melting range of less than 15° C.

26. The wax composition of claim 16, wherein the 90 weight percent of the hydrocarbon wax molecules have a molecular weight ranging from 630 to 800 grams/mole.

27. The wax composition of claim 26, wherein the hydrocarbon wax molecules comprise olefinic hydrocarbon wax molecules and the hydrocarbon wax molecules have a DSC melting point ranging from 70 to 100° C. and DSC melting range of less than 15° C.

28. The wax composition of claim 26, wherein the hydrocarbon wax molecules consist essentially of saturated hydrocarbon wax molecules and the hydrocarbon wax molecules have a DSC melting point ranging from 85 to 105° C. and a DSC melting range of less than 15° C.
